(19) United States
(12) Patent Application Publication
(10) Pub. No.: US 2015/0166670 A1
(43) Pub. Date: Jun. 18, 2015

(54) MULTISPECIFIC ANTIBODIES

(71) Applicant: Hoffmann-La Roche Inc., Little Falls, NJ (US)

(72) Inventors: RAFFAELLA CASTOLDI, MUENCHEN (DE); ALEXANDER HAAS, MUENCHEN (DE); CHRISTIAN KLEIN, BONSTETTEN (CH); WOLFGANG SCHAEFER, MANNHEIM (DE); CLAUDIO SUSTMANN, MUENCHEN (DE)

(73) Assignee: HOFFMANN-LA ROCHE INC., Little Falls, NJ (US)

(21) Appl. No.: 14/551,957

(22) Filed: Nov. 24, 2014

Related U.S. Application Data
(63) Continuation of application No. PCT/EP2013/060529, filed on May 22, 2013.

(30) Foreign Application Priority Data
May 24, 2012 (EP) 12169340.2

(51) Int. Cl. C07K 16/32; C07K 16/46; C07K 16/22

(52) U.S. Cl. CPC C07K 16/32; C07K 16/22; C07K 16/468; C07K 2317/31; C07K 2317/515; C07K 2317/51

(57) ABSTRACT

The present invention relates to bivalent, multispecific antibodies, especially bivalent, trispecific antibodies which bind to human HER1, human HER2, and human HER3, their manufacture and use.

### CROSS REFERENCE TO RELATED APPLICATIONS

This application is a continuation of International Application No. PCT/EP2013/060529 having an international filing date of May 22, 2013, the entire contents of which are incorporated herein by reference, and which claims benefit under 35 U.S.C. §119 to European Patent Application No. 12169340.2, filed May 24, 2012.

### SEQUENCE LISTING

The instant application contains a Sequence Listing submitted via EFS-Web. Said ASCII copy, created on Nov. 24, 2014, is named P31005_US_C_SeqList.txt., and is 68,575 bytes in size.

The present invention relates to novel bivalent, multispecific antibodies, especially tri- or tetraspecific antibodies, especially bivalent, trispecific antibodies which bind to human HER1, human HER2, and human HER3, their manufacture and use.

### BACKGROUND OF THE INVENTION

Engineered proteins, such as bispecific antibodies capable of binding two different antigens are known in the art. Such bispecific binding proteins can be generated using cell fusion, chemical conjugation, or recombinant DNA techniques.

A wide variety of recombinant bispecific antibody formats have been developed in the recent past, e.g. tetravalent bispecific antibodies by fusion of, e.g. an IgG antibody format and single chain domains (see e.g. Coloma, M. J., et al., Nature Biotech. 15 (1997) 159-163; WO 2001/077342; and Morrison, S. L., Nature Biotech. 25 (2007) 1233-1234.

Also several other new formats wherein the antibody core structure (IgA, IgD, IgE, IgG or IgM) is no longer retained such as dia-, tria- or tetrabodies, minibodics, several single chain formats (scFv, Bis-scFv), which are capable of binding two or more antigens, have been developed (Holliger, P., et al., Nature Biotech. 23 (2005) 1126-1136; Fischer, N., and Léger, O., Pathobiology 74 (2007) 3-14; Shen, J., et al., J. Immunol. Methods 318 (2007) 65-74; Wu, C., et al., Nature Biotech. 25 (2007) 1290-1297).

All such formats use linkers either to fuse the antibody core (IgA, IgD, IgE, IgG or IgM) to a further binding protein (e.g. scFv) or to fuse e.g. two Fab fragments or scFv (Fischer, N., and Leger, O., Pathobiology 74 (2007) 3-14). While it is obvious that linkers have advantages for the engineering of bispecific antibodies, they may also cause problems in therapeutic settings. Indeed, these foreign peptides might elicit an immune response against the linker itself or the junction between the protein and the linker. Further more, the flexible nature of these peptides makes them more prone to proteolytic cleavage, potentially leading to poor antibody stability, aggregation and increased immunogenicity. In addition one may want to retain effector functions, such as e.g. complement-dependent cytotoxicity (CDC) or antibody dependent cellular cytotoxicity (ADCC), which are mediated through the Fc-part by maintaining a high degree of similarity to naturally occurring antibodies.

Thus, ideally, one should aim at developing bispecific antibodies that are very similar in general structure to naturally occurring antibodies (like IgA, IgD, IgE, IgG or IgM) with minimal deviation from human sequences.

WO 2009/080251, WO 2009/080252, WO 2009/080253; WO 2009/080254 and Schaefer, et al PNAS 108 (2011) 11187-11192 relate to bispecific bivalent antibodies.

WO 2008/027236; WO 2010/108127 and Bostrom, J., et al., Science 323 (2009) 1610-1614 relate to methods of diversifying the variable heavy chain and light chain domains VH and VL to introduce dual specificities. WO 2010/136172 relates to tri- or tetraspecific antibodies, which however are tri- or tetravalent, WO 2007/146959 relates to pan-cell surface receptor-specific therapeutics

This techniques are not appropriate as a basis for easily developing recombinant, multispecific antibodies against three or four antigens with a IgG-like structure and and IgG-like molecular weight. So far it was not possible to generate a bivalent, tri- or tetraspecific antibody, with a structure similar to naturally occurring bivalent antibodies without further fused binding domains.

### SUMMARY OF THE INVENTION

The invention relates to a multispecific antibody, comprising:
A) a) the light chain and heavy chain of a full length antibody which specifically binds to a first antigen and second antigen; and
   b) the modified light chain and modified heavy chain of a full length antibody which specifically binds to a third antigen, wherein the variable domains VL and VH are replaced by each other, and/or wherein the constant domains CL and CH1 are replaced by each other;
or
B) a) the light chain and heavy chain of a full length antibody which specifically binds to a first antigen; and
   b) the modified light chain and modified heavy chain of a full length antibody which specifically binds to a second antigen and third antigen, wherein the variable domains VL and VH are replaced by each other, and/or wherein the constant domains CL and CH1 are replaced by each other;
or
C) a) the light chain and heavy chain of a full length antibody which specifically binds to a first antigen and a second antigen; and
   b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
In one embodiment the multispecific antibody is characterized in that the antibody is a bivalent, tri- or tetraspecific antibody.
In one embodiment the multispecific antibody is characterized in that the antibody is trispecific and comprises
   A) a) the light chain and heavy chain of a full length antibody which specifically binds to a first antigen and second antigen; and
      b) the modified light chain and modified heavy chain of a full length antibody which specifically binds to a third antigen, wherein the variable domains VL and VH are replaced by each other, and/or wherein the constant domains CL and CH1 are replaced by each other;
   or
   B) a) the light chain and heavy chain of a full length antibody which specifically binds to a first antigen; and
      b) the modified light chain and modified heavy chain of a full length antibody which specifically binds to a second antigen and third antigen, wherein the variable domains VL and VH are replaced by each other, and/or wherein the constant domains CL and CH1 are replaced by each other.
In one embodiment the multispecific antibody is characterized in that
   under A) the first antigen is human HER1, the second antigen human HER3 and the third antigen is human HER2; or
   under B) the first antigen is human HER2, the second antigen human HER1 and the third antigen is human HER3.
In one embodiment the multispecific antibody is characterized in that under A) the first antigen is human HER1, the second antigen human HER3 and the third antigen is human cMET; or
   under B) the first antigen is human cMET, the second antigen human HER1 and the third antigen is human HER3.
In one embodiment the multispecific antibody is characterized in that the antibody is tetraspecific and comprises
   a) the light chain and heavy chain of a full length antibody which specifically binds to a first antigen and a second antigen; and
   b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
In one embodiment the multispecific antibody is characterized in that in the modified light chain and modified heavy chain under b) the variable domains VL and VH are replaced by each other; and wherein the constant domains CL and CH1 are replaced by each other.
In one embodiment the multispecific antibody is characterized in that in the modified light chain and modified heavy chain under b) (only) the variable domains VL and VH are replaced by each other,
In one embodiment the multispecific antibody is characterized in that in the modified light chain and modified heavy chain under b) (only) the constant domains CL and CH1 are replaced by each other.
In one embodiment the multispecific antibody is characterized in that
   the CH3 domain of the heavy chain of the full length antibody of a) and
   the CH3 domain of the modified heavy chain of the full length antibody of b) each meet at an interface which comprises an original interface between the antibody CH3 domains;
      wherein said interface is altered to promote the formation of the trispecific or tetraspecific antibody, wherein the alteration is characterized in that:
      i) the CH3 domain of one heavy chain is altered,
      so that within the original interface the CH3 domain of one heavy chain that meets the original interface of the CH3 domain of the other heavy chain within the tri- or tetraspecific antibody,
      an amino acid residue is replaced with an amino acid residue having a larger side chain volume, thereby generating a protuberance within the interface of the CH3 domain of one heavy chain which is positionable in a cavity within the interface of the CH3 domain of the other heavy chain
      and
      ii) the CH3 domain of the other heavy chain is altered, so that within the original interface of the second CH3 domain that meets the original interface of the first CH3 domain within the tri- or tetraspecific antibody
      an amino acid residue is replaced with an amino acid residue having a smaller side chain volume, thereby generating a cavity within the interface of the second CH3 domain within which a protuberance within the interface of the first CH3 domain is positionable.
In one embodiment the multispecific antibody is characterized in that the said amino acid residue having a larger side chain volume is selected from the group consisting of arginine (R), phenylalanine (F), tyrosine (Y), tryptophan (W) and said amino acid residue having a smaller side chain volume is selected from the group consisting of alanine (A), serine (S), threonine (T), valine (V).
In one embodiment the multispecific antibody is characterized in that both CH3 domains are further altered by the introduction of cysteine (C) as amino acid in the corresponding positions of each CH3 domain such that a disulfide bridge between both CH3 domains can be formed.
A further embodiment of the invention is a method for the preparation of a multispecific antibody according to the invention
   comprising the steps of
   a) transforming a host cell with vectors comprising nucleic acid molecules encoding
      A) a) the light chain and heavy chain of a full length antibody which specifically binds to a first antigen and second antigen; and
         b) the modified light chain and modified heavy chain of a full length antibody which specifically binds to a third antigen, wherein the variable domains VL and VH are replaced by each other, and/or wherein the constant domains CL and CH1 are replaced by each other;
      or
      B) a) the light chain and heavy chain of a full length antibody which specifically binds to a first antigen; and
         b) the modified light chain and modified heavy chain of a full length antibody which specifically binds to a second antigen and third antigen, wherein the variable domains VL and VH are replaced by each other, and/or wherein the constant domains CL and CH1 are replaced by each other;
      or
      C) a) the light chain and heavy chain of a full length antibody which specifically binds to a first antigen and a second antigen; and
         b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
   b) culturing the host cell under conditions that allow synthesis of said antibody molecule; and
   c) recovering said antibody molecule from said culture.

The invention further comprises nucleic acid encoding the multispecific antigen binding protein according to the invention.

The invention further comprises vectors comprising nucleic acid encoding the multispecific antigen binding protein according to the invention.

A further embodiment of the invention is a host cell comprising
vectors comprising nucleic acid molecules encoding
A) a) the light chain and heavy chain of a full length antibody which specifically binds to a first antigen and second antigen; and
   b) the modified light chain and modified heavy chain of a full length antibody which specifically binds to a third antigen, wherein the variable domains VL and VH are replaced by each other, and/or wherein the constant domains CL and CH1 are replaced by each other;
or
B) a) the light chain and heavy chain of a full length antibody which specifically binds to a first antigen; and
   b) the modified light chain and modified heavy chain of a full length antibody which specifically binds to a second antigen and third antigen, wherein the variable domains VL and VH are replaced by each other, and/or wherein the constant domains CL and CH1 are replaced by each other;
or
C) a) the light chain and heavy chain of a full length antibody which specifically binds to a first antigen and a second antigen; and
   b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.

A further embodiment of the invention is a composition, preferably a pharmaceutical or a diagnostic composition of the antibody according to the invention.+

A further embodiment of the invention is a pharmaceutical composition comprising an antibody according to the invention and at least one pharmaceutically acceptable excipient.

A further embodiment of the invention is a method for the treatment of a patient in need of therapy, characterized by administering to the patient a therapeutically effective amount of an antibody according to the invention.

This is the first time that multispecific antibodies against three or four antigens with a IgG-like structure and IgG-like molecular weight are provided.

According to the invention, the ratio of a desired multispecific antibody compared to undesired side products can be improved by the replacement of certain domains in only one pair of heavy chain and light chain (HC/LC) of the two full length antibody arms (e.g. replacement/exchange of the VH domain and the VL domain, or replacement/exchange the CH1 domain and the CL domain; or replacement/exchange of both the VH and CH1 domain and the VH and VL domain). In this way the undesired mispairing of the light chain with the wrong heavy chain leads to undesired dysfunctional by products (misparing of VH¹ with VH² and/or VH² with VH¹) can be reduced (see FIG. 3)

### DESCRIPTION OF THE FIGURES

FIG. 1: Schematic structure of a full length antibody without CH4 domain specifically binding to a first antigen 1 with two pairs of heavy and light chain which comprise variable and constant domains in a typical order.

FIG. 2a: Schematic structure of a trispecific antibody, comprising:
a) the light chain and heavy chain of a full length antibody (e.g. with diversified VH¹ and VL¹) which specifically binds to a first antigen and second antigen; and
b) the modified light chain and modified heavy chain of a full length antibody (with VH² and VL²) which specifically binds to a third antigen, wherein the constant domains CL and CH1 are replaced by each other.

FIG. 2b: Schematic structure of a trispecific antibody, comprising:
a) the light chain and heavy chain of a full length antibody (e.g. with diversified VH¹ and VL¹) which specifically binds to a first antigen and second antigen; and
b) the modified light chain and modified heavy chain of a full length antibody (with VH² and VL²) which specifically binds to a third antigen, wherein the variable domains VL and VH are replaced by each other and the constant domains CL and CH1 are replaced by each other;
   with exemplary CH3 modifications in both heavy chains (“knobs-into-hole”)

FIG. 2c: Schematic structure of a trispecific antibody, comprising:
a) the light chain and heavy chain of a full length antibody (with VH¹ and VL¹) which specifically binds to a first antigen and;
b) the modified light chain and modified heavy chain of a full length antibody (e.g. with diversified VH² and VL²) which specifically binds to a second antigen and third antigen, wherein the variable domains VL and VH are replaced by each other;
   with exemplary CH3 modifications in both heavy chains (“knobs-into-hole”)

FIG. 2d: Schematic structure of a tetraspecific antibody, comprising:
a) the light chain and heavy chain of a full length antibody (e.g. with diversified VH¹ and VL¹) which specifically binds to a first antigen and a second antigen and; and
b) the modified light chain and modified heavy chain of a full length antibody (e.g. with diversified VH² and VL²) which specifically binds to a third antigen and fourth antigen, wherein the variable domains VL and VH are replaced by each other, and/or wherein the constant domains CL and CH1 are replaced by each other.

FIG. 3a: Schematic summary of the dual-affinity-crossmab principle. The crossover technology was used for the heavy chain, light chain combination which recognizes one antigen on one Fab arm.

FIG. 3b: The crossover technology was used for the heavy chain, light chain combination which recognizes two antigens on one Fab arm. The knobs-into holes technology including disulfide stabilization (heavy chain 1: S354C, T366W; heavy chain 2: T366S, L368A, Y407V, Y349C) can be used for either combination.

FIG. 4: Schematic structure of the undesired dysfunctional side products due to light and heavy chain mispairing (leading to mispaired VH1 with VH2 and/or VH2 with VH1)

FIG. 5a: Schematic presentation of the eukaryotic expression vector used for cloning of the heavy chain constructs.

FIG. 5b: Schematic presentation of the eukaryotic vector used for cloning of the light chain constructs.

FIG. 6a: Results of analytical HPLC of the VEGF-Her2-DAF test expression. (A, C, E) Biological replicate 1 and (B, D, F) biological replicate 2 (K:H=knob to hole ratio of transfected plasmids) of protein A immuno-precipitated material.

FIG. 6b: SDS-PAGE of VEGF-Her2-DAF expressions. Two equal samples represent analysis of technical replicates (NR, non-reducing conditions; Red, reducing conditions) of protein A immuno-precipitated material

FIG. 6c: Marker proteins correlating elution time and size in analytical HPLC.

FIG. 7a: Results of analytical HPLC of the VEGF-Her2-DAF-xAng2 test expression. (A, C, E) Biological replicate 1 and (B, D, F) biological replicate 2 (K:H=knob to hole ratio of transfected plasmids) of protein A immuno-precipitated material.

FIG. 7b: SDS-PAGE of VEGF-Her2-DAF-xAng2 expressions. Two equal samples represent analysis of technical replicates (NR, non-reducing conditions; Red, reducing conditions) of protein A immuno-precipitated material.

FIG. 8a: Results of analytical HPLC of the VEGF-Her2-DAF-xHer1-Her3 DAF test expression. (A, B) Biological replicate 1 and 2.

FIG. 8b: SDS-PAGE of VEGF-Her2-DAF-xHer1-Her3 DAF expressions. (A,B) are the replicate analyses of the analytical HPLC presented in A (NR, non-reducing conditions; Red, reducing conditions).

FIG. 9: SDS-PAGE of KiH Her1-Her3 DAF-xHer2 expressions (NR, non-reducing conditions; Red, reducing conditions).

FIG. 10a: Proliferation assay with trispecific antibody KiH Her1-Her3-DAF-xHer2. A431 were incubated with 30 μg/mL of trispecific antibody or control IgG antibody. 5 days post-antibody addition an ATP-release assay was performed (Cell Titer Glow, Promega).

FIG. 10b: Proliferation assay with trispecific antibody KiH Her1-Her3 DAF-xHer2. A431 were incubated with 30 μg/mL of indicated antibodies. 5 days post-antibody addition an ATP-release assay was performed (Cell Titer Glow, Promega).

FIG. 11a: Proliferation assay with trispecific antibody KiH Her1-Her3 DAF-xHer2. MDA-MB-175 VII cells were incubated with a dilution series of the trispecific antibody KiH Her1-Her3 DAF-xHer2 or control IgG antibody. 5 days post-antibody addition an ATP-release assay was performed (Cell Titer Glow, Promega).

FIG. 11b: Proliferation assay with trispecific antibody KiH Her1-Her3 DAF-xHer2. MDA-MB-175 VII cells were incubated with a dilution series of the trispecific antibody KiH Her1-Her3 DAF-xHer2 or control IgG antibody. 5 days post antibody addition an ATP-release assay was performed.

FIG. 12a: Binding kinetics of KiH Her1-Her3 DAF-xHer2 or respective parental antibodies. (A, B, C) 1^{st} and 2^{nd} inject indicate the order of ErbB receptor ectodomain addition.

FIG. 12b: Binding kinetics of KiH Her1-Her3 DAF-xHer2 or respective parental antibodies. 1^{st} and 2^{nd} inject indicate the order of ErbB receptor ectodomain addition.

FIG. 12c: Binding kinetics of KiH Her1-Her3 DAF-xHer2 or respective parental antibodies. 1^{st} and 2^{nd} inject indicate the order of ErbB receptor ectodomain addition.

FIG. 13: ADCC Induction by trispecific Her1-Her3 DAF-xHer2 antibody in A431 epidermoid cancer cells.
Image width of a single panel is 170.83 μm
Upper row corresponds to CMFDA labeled tumor cells (normally displayed in the green channel)
Lower row corresponds to PKH26 labelled natural killer cells (normally displayed in the red channel)

### DETAILED DESCRIPTION OF THE INVENTION

The invention relates to a multispecific antibody, comprising:
A) a) the light chain and heavy chain of a full length antibody which specifically binds to a first antigen and second antigen; and
   b) the modified light chain and modified heavy chain of a full length antibody which specifically binds to a third antigen, wherein the variable domains VL and VH are replaced by each other, and/or wherein the constant domains CL and CH1 are replaced by each other;
or
B) a) the light chain and heavy chain of a full length antibody which specifically binds to a first antigen; and
   b) the modified light chain and modified heavy chain of a full length antibody which specifically binds to a second antigen and third antigen, wherein the variable domains VL and VH are replaced by each other, and/or wherein the constant domains CL and CH1 are replaced by each other;
or
C) a) the light chain and heavy chain of a full length antibody which specifically binds to a first antigen and a second antigen; and
   b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
In one embodiment the multispecific antibody is characterized in that the antibody is trispecific and comprises
   A) a) the light chain and heavy chain of a full length antibody which specifically binds to a first antigen and second antigen; and
      b) the modified light chain and modified heavy chain of a full length antibody which specifically binds to a third antigen, wherein the variable domains VL and VH are replaced by each other, and/or wherein the constant domains CL and CH1 are replaced by each other;
   or
   B) a) the light chain and heavy chain of a full length antibody which specifically binds to a first antigen; and
      b) the modified light chain and modified heavy chain of a full length antibody which specifically binds to a second antigen and third antigen, wherein the variable domains VL and VH are replaced by each other, and/or wherein the constant domains CL and CH1 are replaced by each other.
In one embodiment the multispecific antibody is characterized in that the antibody is tetraspecific and comprises
   a) the light chain and heavy chain of a full length antibody which specifically binds to a first antigen and a second antigen; and
   b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
In one embodiment the multispecific antibody is characterized in that in the modified light chain and modified heavy chain under b) the variable domains VL and VH are replaced by each other; and wherein the constant domains CL and CH1 are replaced by each other.
In one embodiment the multispecific antibody is characterized in that in the modified light chain and modified heavy chain under b) (only) the variable domains VL and VH are replaced by each other,
In one embodiment the multispecific antibody is characterized in that in the modified light chain and modified heavy chain under b) (only) the constant domains CL and CH1 are replaced by each other.

According to the invention, the ratio of a desired multispecific antibody compared to undesired side products (due to mispairing of the light chain with the “wrong” heavy chain of the antibody which specifically binds to the other antigen (see FIG. 3) can be improved by the replacement of certain domains in only one pair of heavy chain and light chain (HC/LC). While the first of the two full length HC/LC pairs is left essentially unchanged, the second of the two full length HC/LC pairs o, and is modified by the following replacement:
light chain: replacement of the variable light chain domain VL by the variable heavy chain domain VH of said antibody which specifically binds to a second antigen, and/or the constant light chain domain CL by the constant heavy chain domain CH1 of said antibody which specifically binds to a second antigen, and
heavy chain: replacement of the variable heavy chain domain VH by the variable light chain domain VL of said antibody which specifically binds to a second antigen, and/or the constant heavy chain domain CH1 by the constant light chain domain CL of said antibody which specifically binds to a second antigen.

Thus the resulting multispecific antibody according to the invention are artificial antibodies which comprise

### A)

a) the light chain and heavy chain of an antibody which specifically binds to a first and a second antigen; and
b) the light chain and heavy chain of an antibody which specifically binds to a third antigen,
   wherein said light chain (of an antibody which specifically binds to a third antigen) contains a variable domain VH instead of VL
   and/or a constant domain CH1 instead of CL
   wherein said heavy chain (of an antibody which specifically binds to a third antigen) contains a variable domain VL instead of VH
   and/or a constant domain CL instead of CH1;
      or

### B)

a) the light chain and heavy chain of an antibody which specifically binds to a first antigen; and
b) the light chain and heavy chain of an antibody which specifically binds to a second and a third antigen,
   wherein said light chain (of an antibody which specifically binds to a second and a third antigen) contains a variable domain VH instead of VL
   and/or a constant domain CH1 instead of CL
   wherein said heavy chain (of an antibody which specifically binds to a second and a third antigen) contains a variable domain VL instead of VH
   and/or a constant domain CL instead of CH1;
      or

### C)

a) the light chain and heavy chain of an antibody which specifically binds to a first and a second antigen; and
b) the light chain and heavy chain of an antibody which specifically binds to a third and a fourth antigen,
   wherein said light chain (of an antibody which specifically binds to a third and a fourth antigen) contains a variable domain VH instead of VL
   and/or a constant domain CH1 instead of CL
   wherein said heavy chain (of an antibody which specifically binds to a third and a fourth antigen) contains a variable domain VL instead of VH
   and/or a constant domain CL instead of CH1.

In an additional aspect of the invention such improved ratio of a desired bivalent, multispecific antibody compared to undesired side products can be further improved by modifications of the CH3 domains of said full length antibodies which specifically bind to a first and second antigen within the tri- or tetraspecific antibody.

Thus in one preferred embodiment of the invention the CH3 domains of said tri- or tetraspecific antibody (in the heavy chain and in the modified heavy) according to the invention can be altered by the “knob-into-holes” technology which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

Thus in one aspect of the invention said trispecific or tetraspecific antibody is further characterized in that the CH3 domain of the heavy chain of the full length antibody of a) and the CH3 domain of the modified heavy chain of the full length antibody of b) each meet at an interface which comprises an original interface between the antibody CH3 domains;

wherein said interface is altered to promote the formation of the trispecific or tetraspecific antibody, wherein the alteration is characterized in that:
i) the CH3 domain of one heavy chain is altered,
so that within the original interface the CH3 domain of one heavy chain that meets the original interface of the CH3 domain of the other heavy chain within the tri- or tetraspecific antibody,
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
   and
ii) the CH3 domain of the other heavy chain is altered,
so that within the original interface of the second CH3 domain that meets the original interface of the first CH3 domain within the tri- or tetraspecific antibody
an amino acid residue is replaced with an amino acid residue having a smaller side chain volume, thereby generating a cavity within the interface of the second CH3 domain within which a protuberance within the interface of the first CH3 domain is positionable.

Preferably said amino acid residue having a larger side chain volume is selected from the group consisting of arginine (R), phenylalanine (F), tyrosine (Y), tryptophan (W).

Preferably said amino acid residue having a smaller side chain volume is selected from the group consisting of alanine (A), serine (S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed.

In one preferred embodiment, said trispecific or tetraspecific antibody comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M., et al., Nature Biotech. 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, said trispecific or tetraspecific antibody comprises Y349C, T366W mutations in one of the two CH3 domains and E356C, T366S, L368A, Y407V mutations in the other of the two CH3 domains or said trispecific or tetraspecific antibody comprises Y349C, T366W mutations in one of the two CH3 domains and S354C, T366S, L368A, Y407V mutations in the other of the two CH3 domains (the additional Y349C mutation in one CH3 domain and the additional E356C or S354C mutation in the other CH3 domain forming a interchain disulfide bridge) (numbering always according to EU index of Kabat). But also other knobs-in-holes technologies as described by EP 1 870 459 A1, can be used alternatively or additionally. A preferred example for said trispecific or tetraspecific antibody are R409D; K370E mutations in the CH3 domain of the “knobs chain” and D399K; E357K mutations in the CH3 domain of the “hole chain” (numbering always according to EU index of Kabat).

In another preferred embodiment said trispecific or tetraspecific antibody comprises a T366W mutation in the CH3 domain of the “knobs chain” and T366S, L368A, Y407V mutations in the CH3 domain of the “hole chain” and additionally R409D; K370E mutations in the CH3 domain of the “knobs chain” and D399K; E357K mutations in the CH3 domain of the “hole chain”.

In another preferred embodiment said trispecific or tetraspecific antibody comprises Y349C, T366W mutations in one of the two CH3 domains and S354C, T366S, L368A, Y407V mutations in the other of the two CH3 domains or said trispecific or tetraspecific antibody comprises Y349C, T366W mutations in one of the two CH3 domains and S354C, T366S, L368A, Y407V mutations in the other of the two CH3 domains and additionally R409D; K370E mutations in the CH3 domain of the “knobs chain” and D399K; E357K mutations in the CH3 domain of the “hole chain”.

In one embodiment the multispecific antibody is characterized in that

under A) the first antigen is human HER1, the second antigen human HER3 and the third antigen is human HER2; or
under B) the first antigen is human HER2, the second antigen human HER1 and the third antigen is human HER3.

In one embodiment the multispecific antibody is characterized in comprising the amino acid sequences of SEQ ID NOs: 4, 9, 13 and 18.

In one embodiment the multispecific antibody is a bivalent, trispecific antibody and comprises a
a) the light chain and heavy chain of a full length antibody which specifically binds to human HER1 and human HER3; and
b) the modified light chain and modified heavy chain of a full length antibody which specifically binds to human HER2, wherein the variable domains VL and VH are replaced by each other, and/or wherein the constant domains CL and CH1 are replaced by each other.

In one embodiment such bivalent, trispecific antibody which specifically binds to human HER1, human HER3, and human HER2 comprises the amino acid sequences of SEQ ID NOs: 4, 9, 13 and 18.

In one embodiment such bivalent, trispecific antibody which specifically binds to human HER1, human HER3, and human HER2 comprises the amino acid sequences of SEQ ID NOs: 4, 9, 13 and 18 and the antibody is characterized by the following properties:

i) the antibody binds to human HER1 (ectodomain ECD) with an affinity of KD 1.7E-08 [M] measured by surface plasmon resonance at 37° C.; and
ii) the antibody binds to human HER2 (ectodomain ECD) with an affinity of KD 4.4E-09 [M] measured by surface plasmon resonance at 37° C.; and
iii) the antibody binds to human HER2 (ectodomain ECD) with an affinity of KD 1.8E-09 [M] measured by surface plasmon resonance at 37° C.; and
iv) the antibody can simultaneously bind to human Her1 and human Her2 or simultaneously bind to human Her3 and human Her2;

In one embodiment such bivalent, trispecific antibody which specifically binds to human HER1, human HER3, and human HER2 comprises the amino acid sequences of SEQ ID NOs: 4, 9, 13 and 18 and the antibody is characterized by one ore more of the following properties:
i) the antibody inhibits growth of MDA-MB-175 breast cancer cells by more than 85% at a concentration of 50 μg/mL;
ii) the antibody inhibits growth of A431 epidermoid cancer cells (which express HER1) by more than 50% at a concentration of 30 μg/mL; and
iii) the antibody induces ADCC in A431 epidermoid cancer cells and thereby eliminating within 2.5 h approximately 100% of the cancer cells;

In one embodiment such bivalent, trispecific antibody which specifically binds to human HER1, human HER3, and human HER2 comprises the amino acid sequences of SEQ ID NOs: 4, 9, 13 and 18 and wherein antibody is glycosylated with a sugar chain at Asn297 (Numbering according to Kabat) whereby the amount of fucose within said sugar chain is 65% or lower (in another embodiment is the amount of fucose within said sugar chain is between 5% and 65%, in one embodiment between 20% and 40%).

In one embodiment the multispecific antibody is characterized in that under A) the first antigen is human HER1, the second antigen human HER3 and the third antigen is human cMET; or under B) the first antigen is human cMET, the second antigen human HER1 and the third antigen is human HER3.

In one embodiment the multispecific antibody is characterized in comprising the amino acid sequences of SEQ ID NOs: 4, 10, 13 and 19.

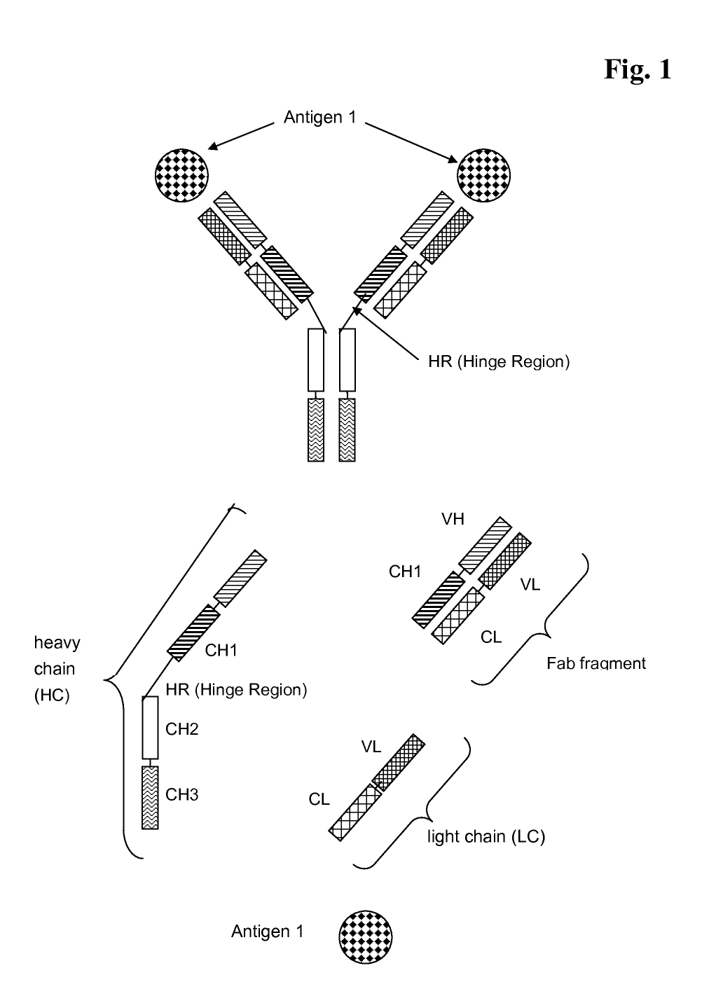

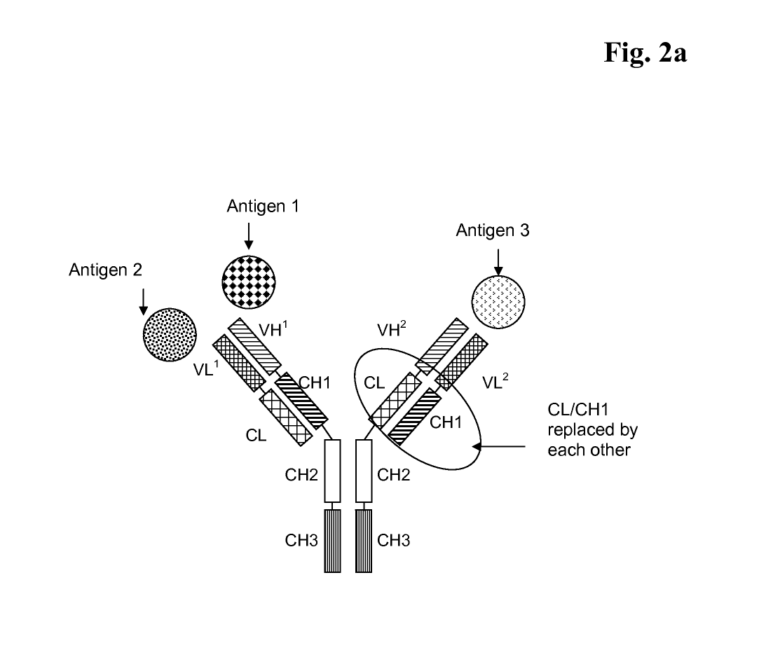

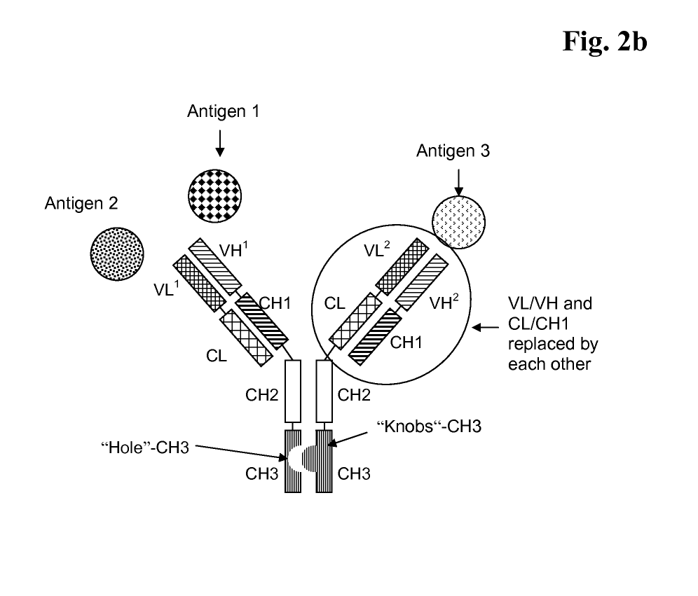

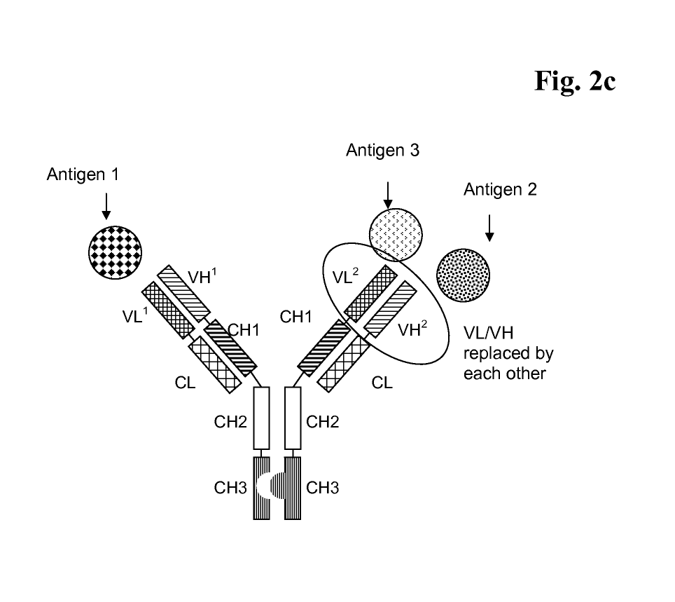

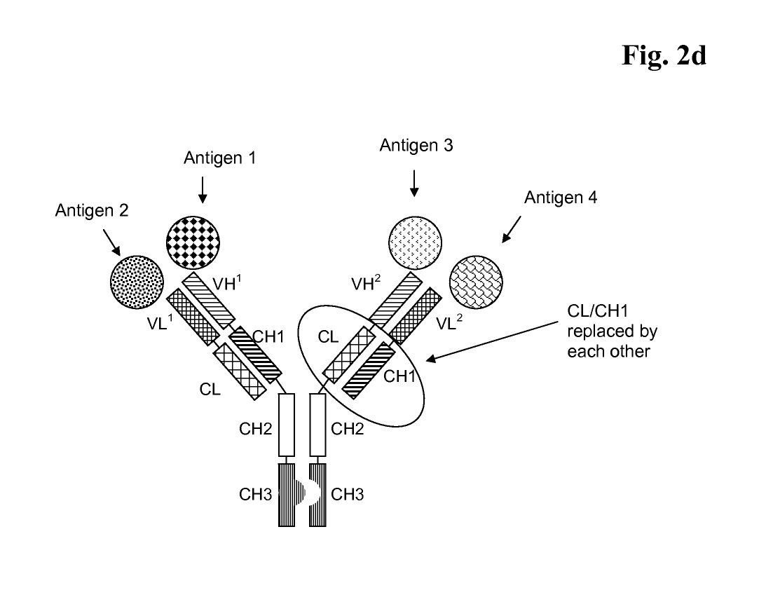

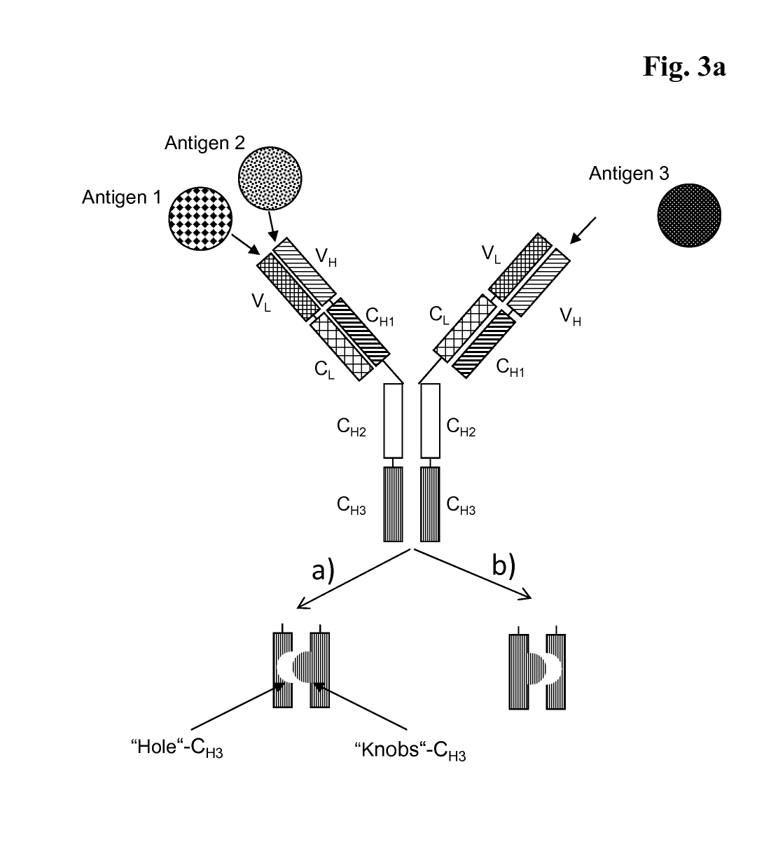

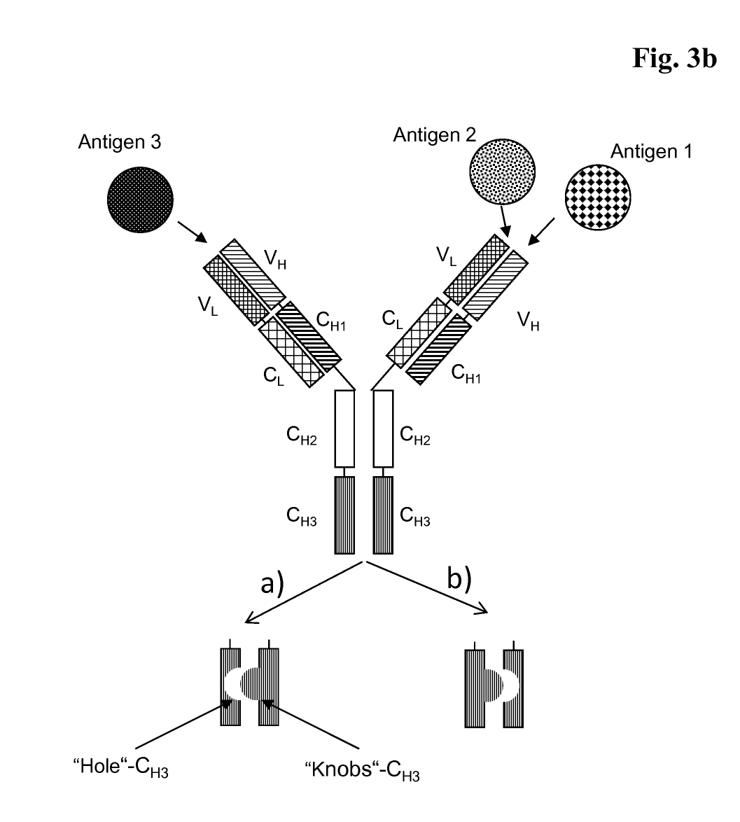

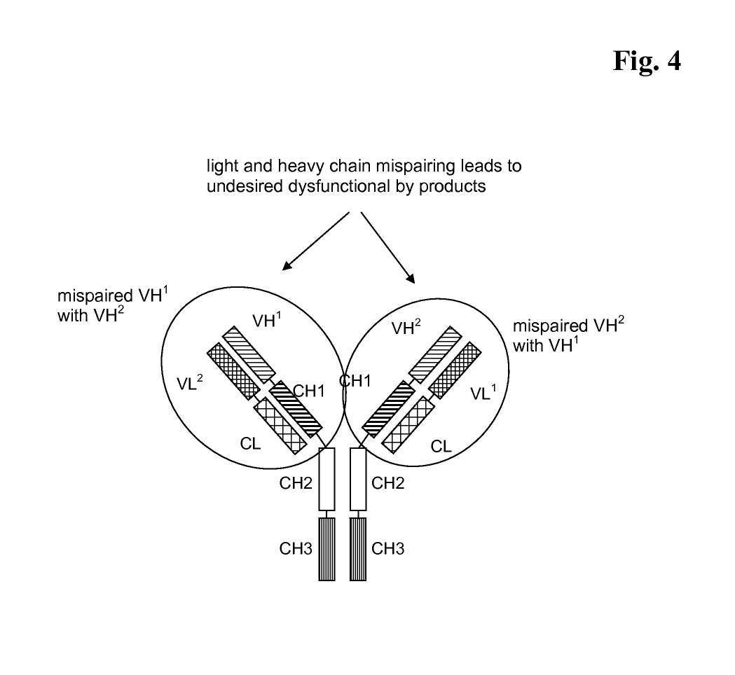

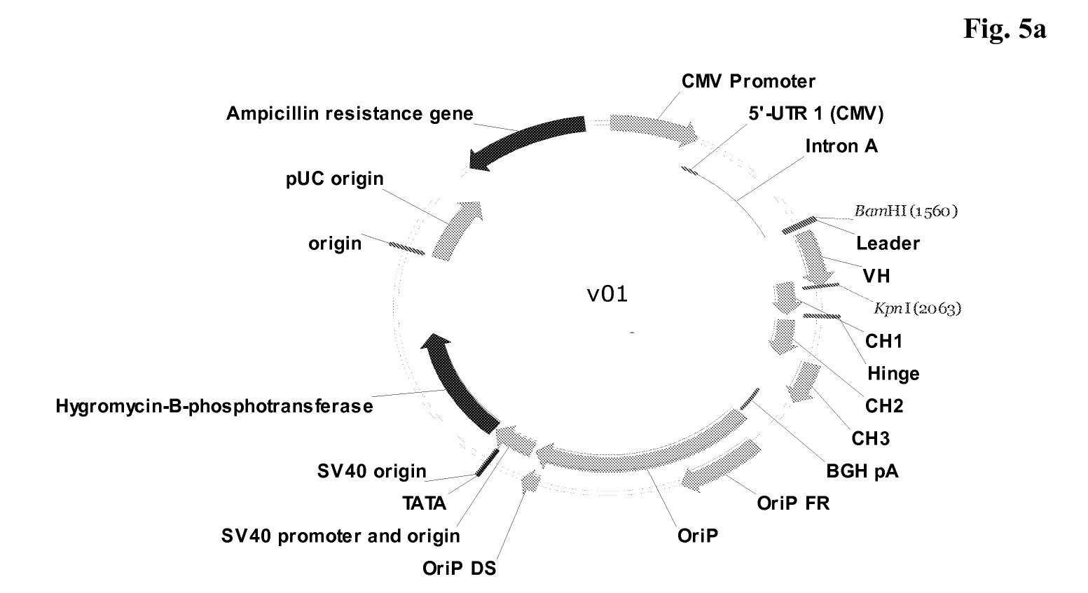

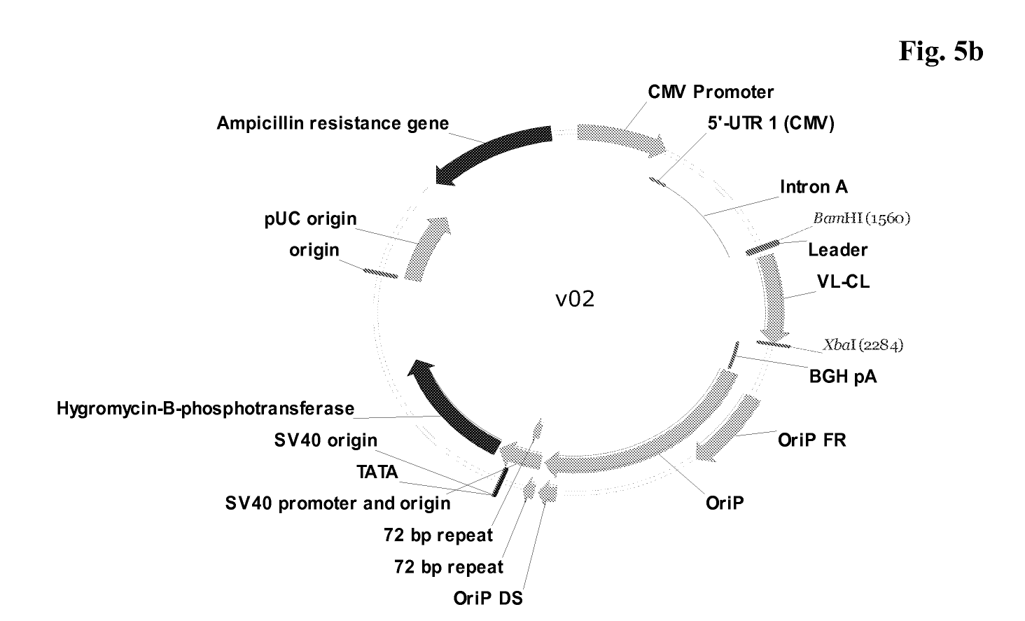

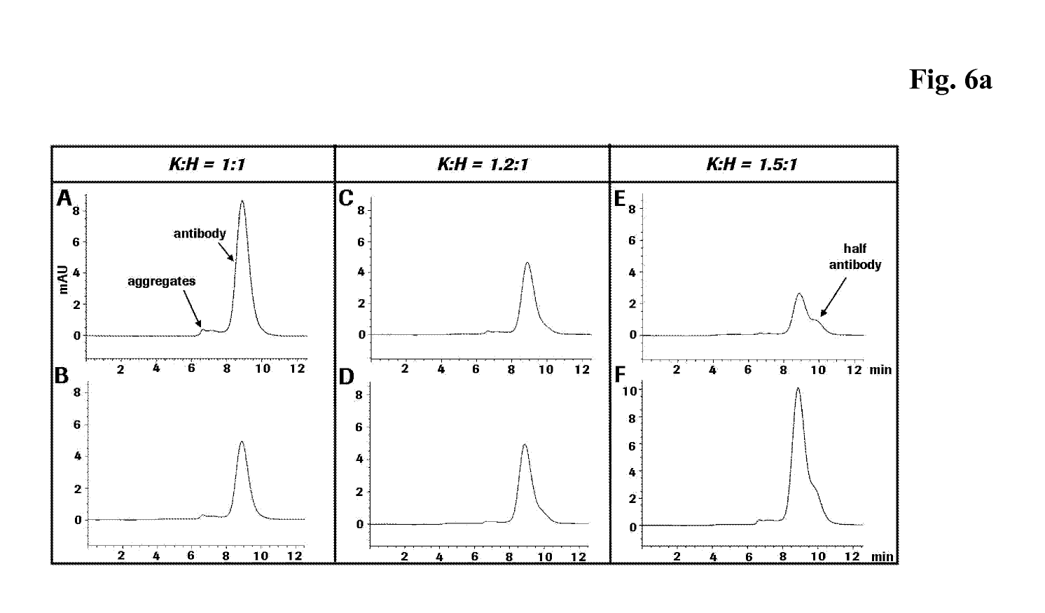

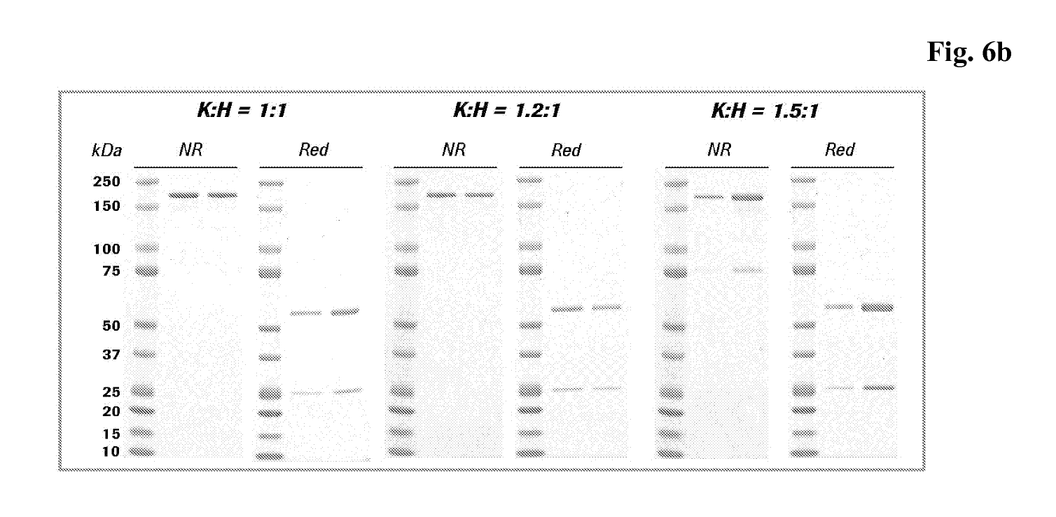

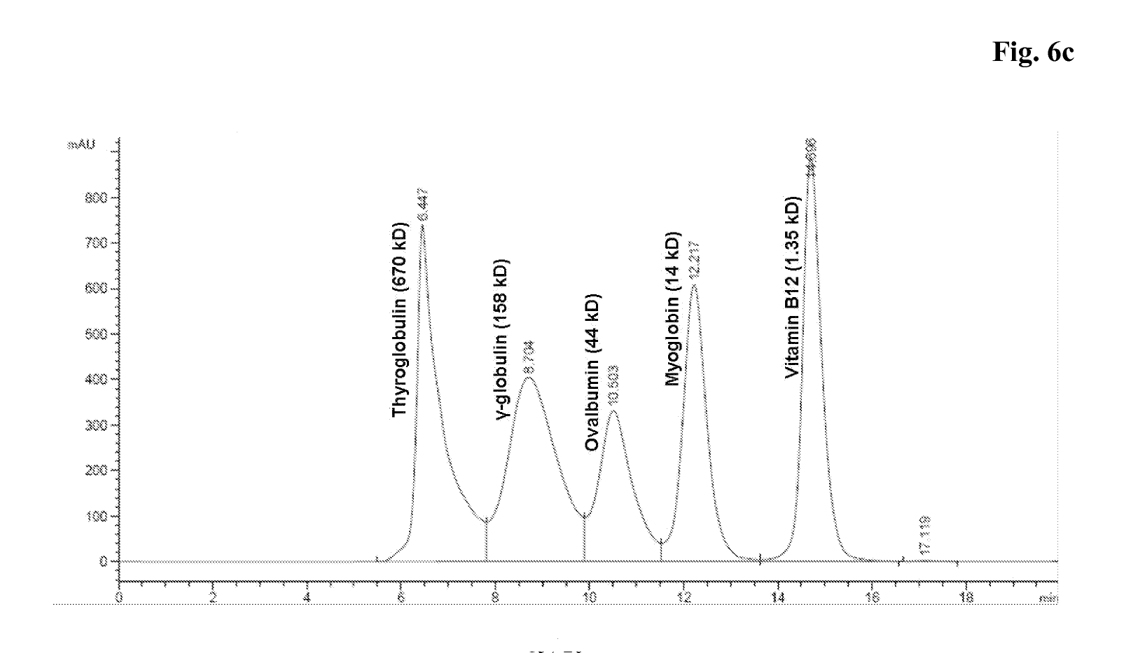

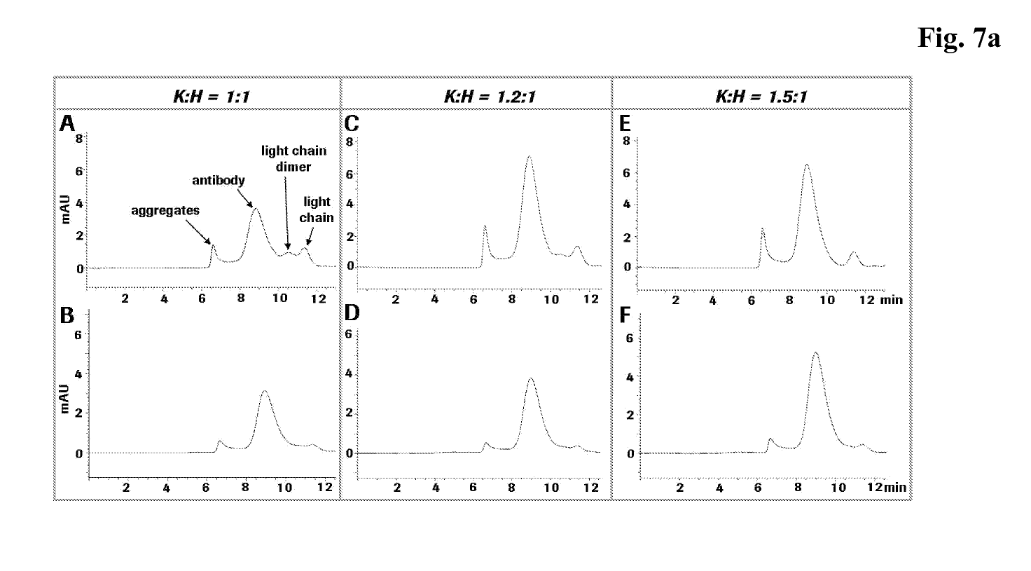

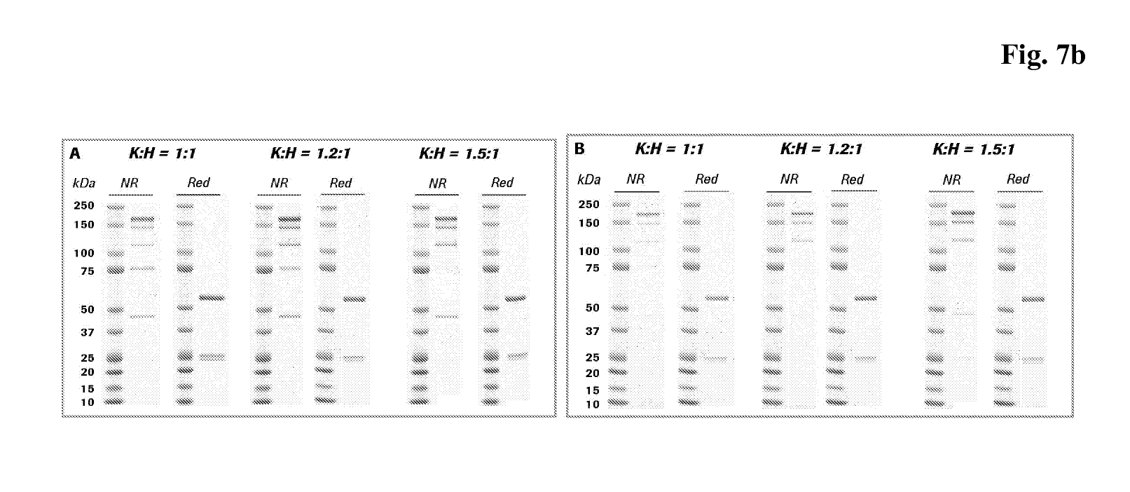

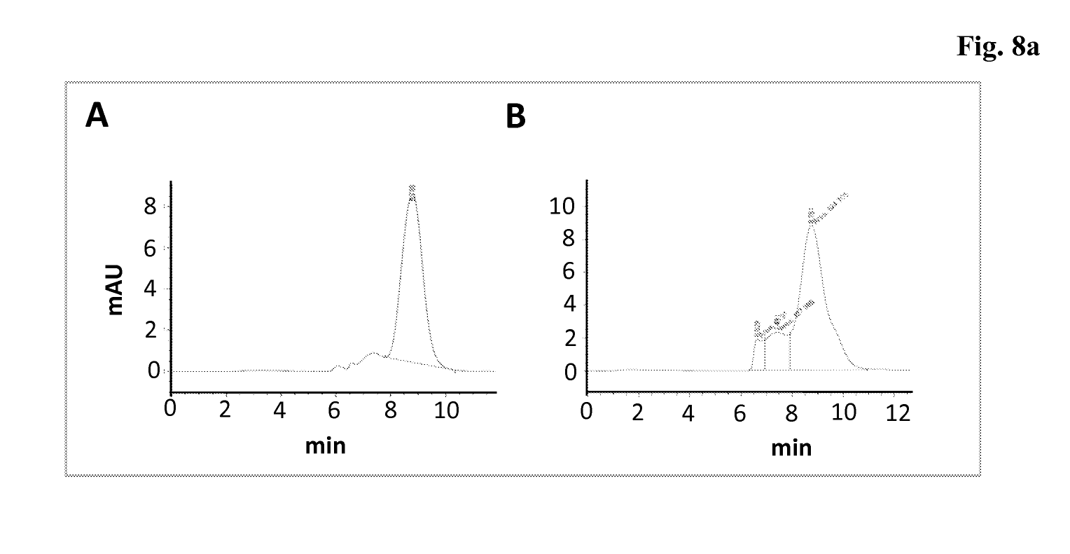

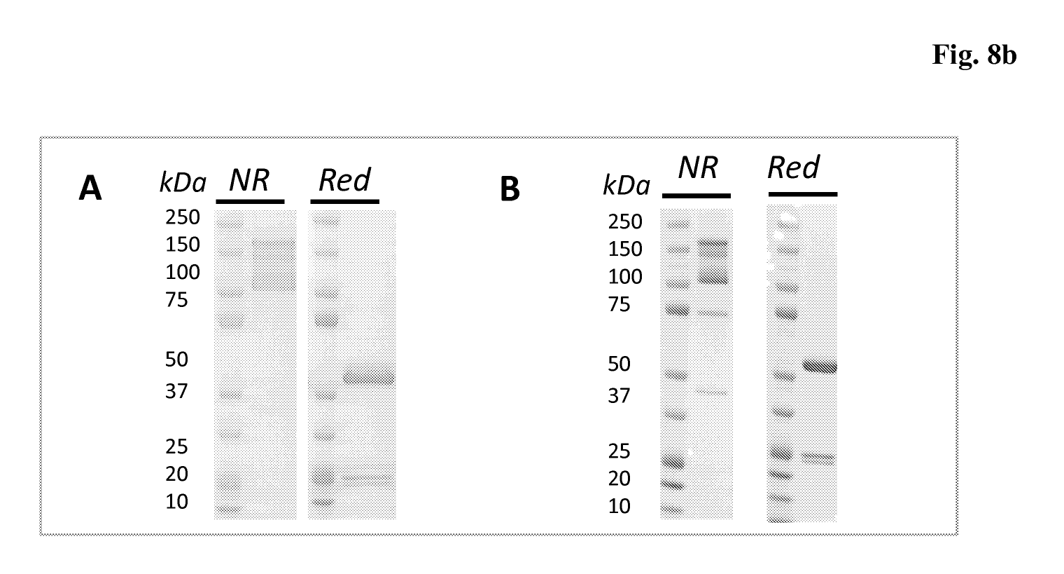

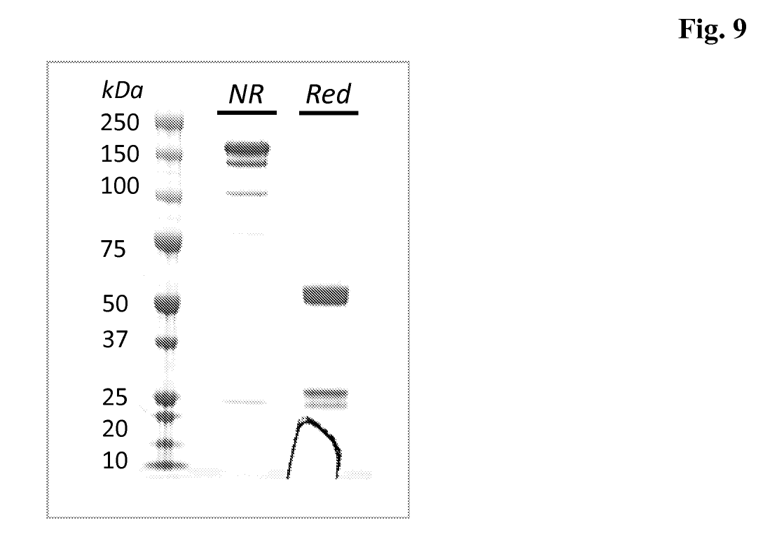

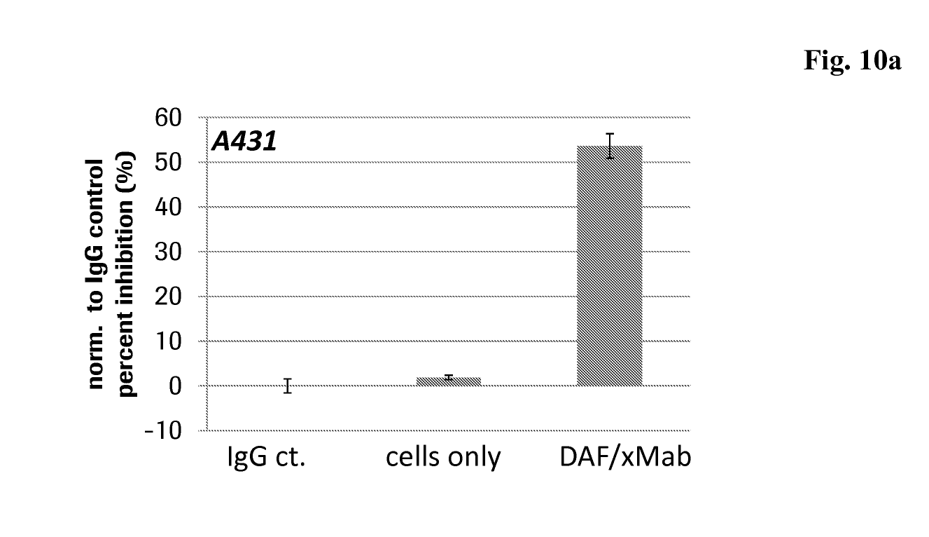

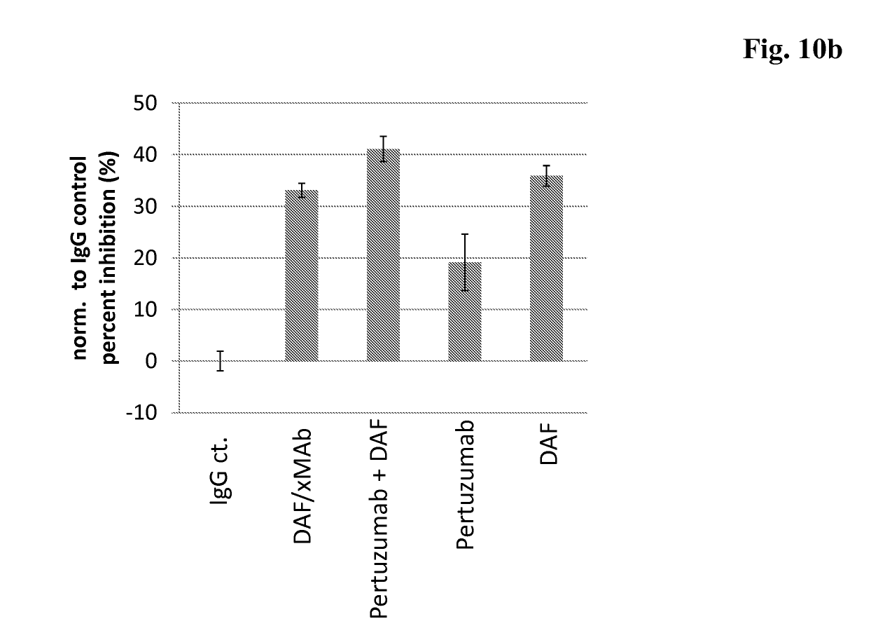

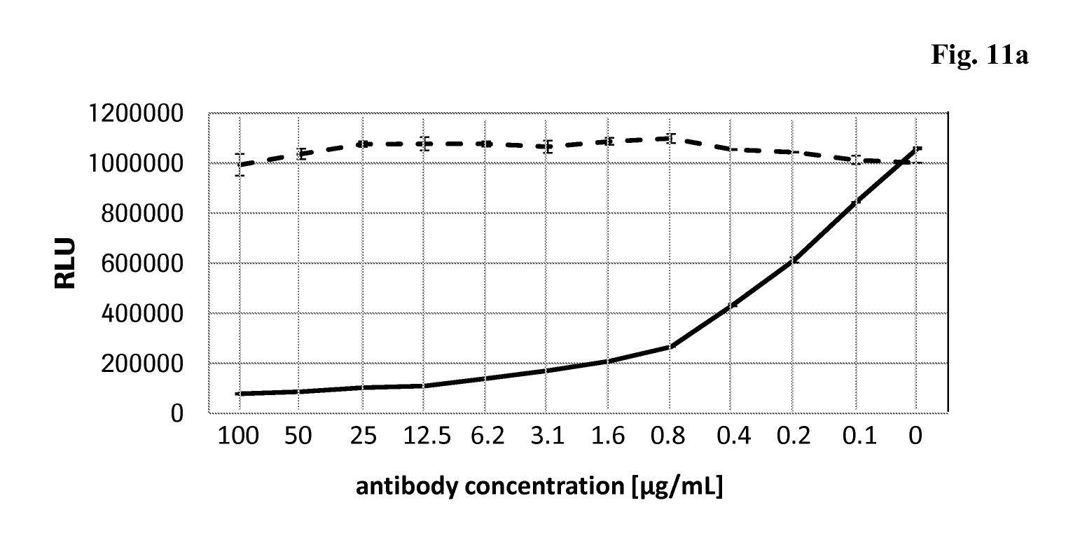

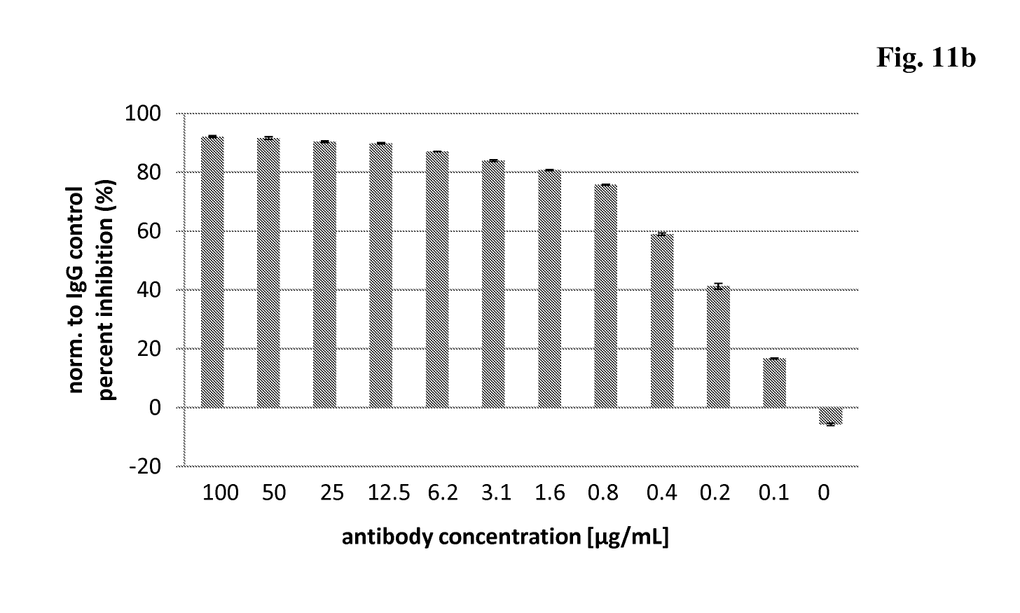

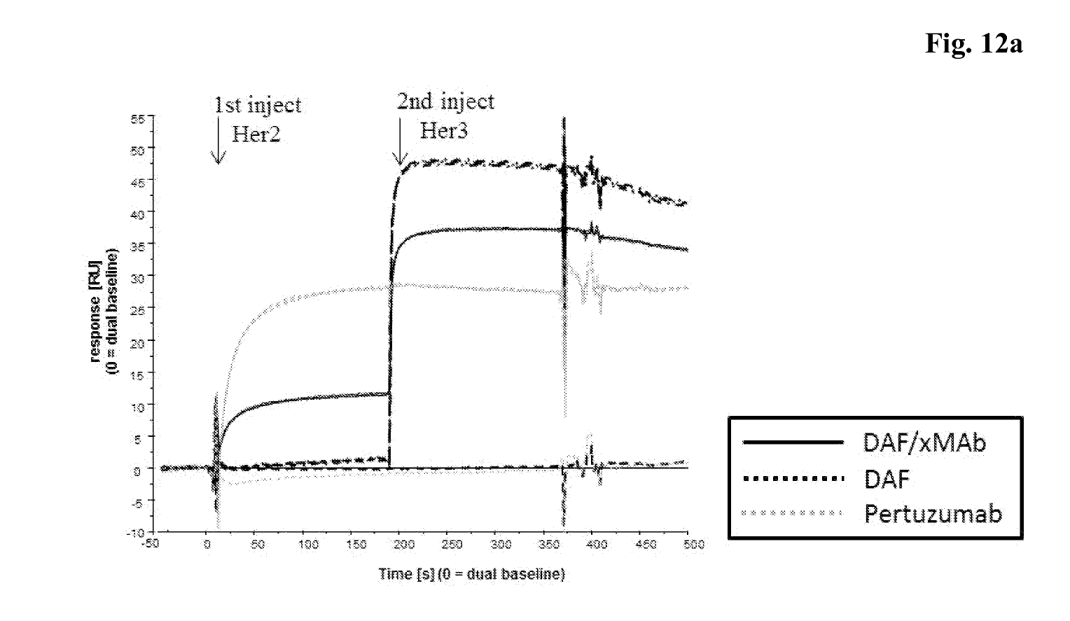

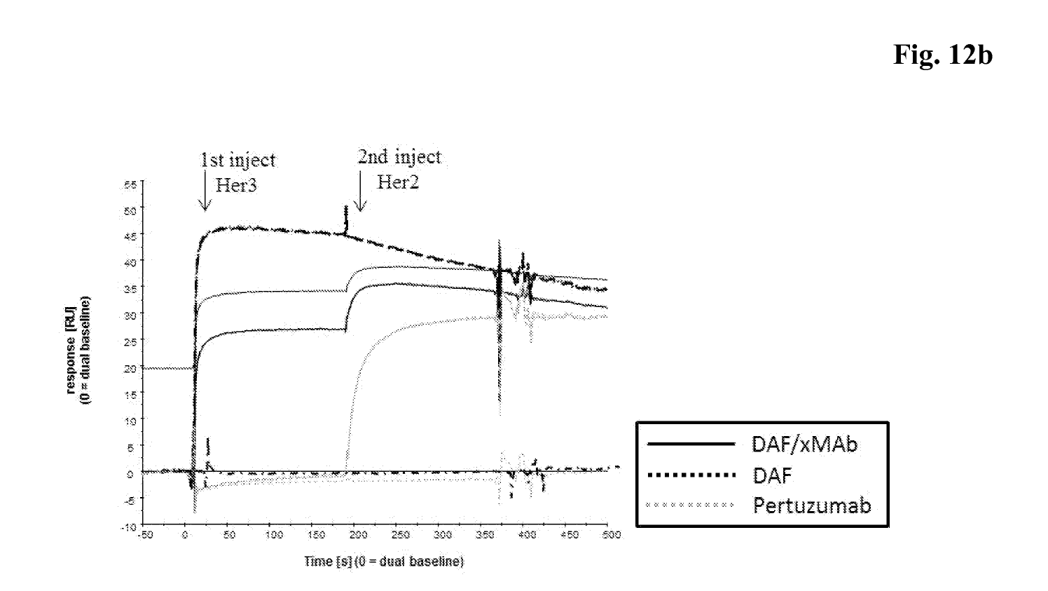

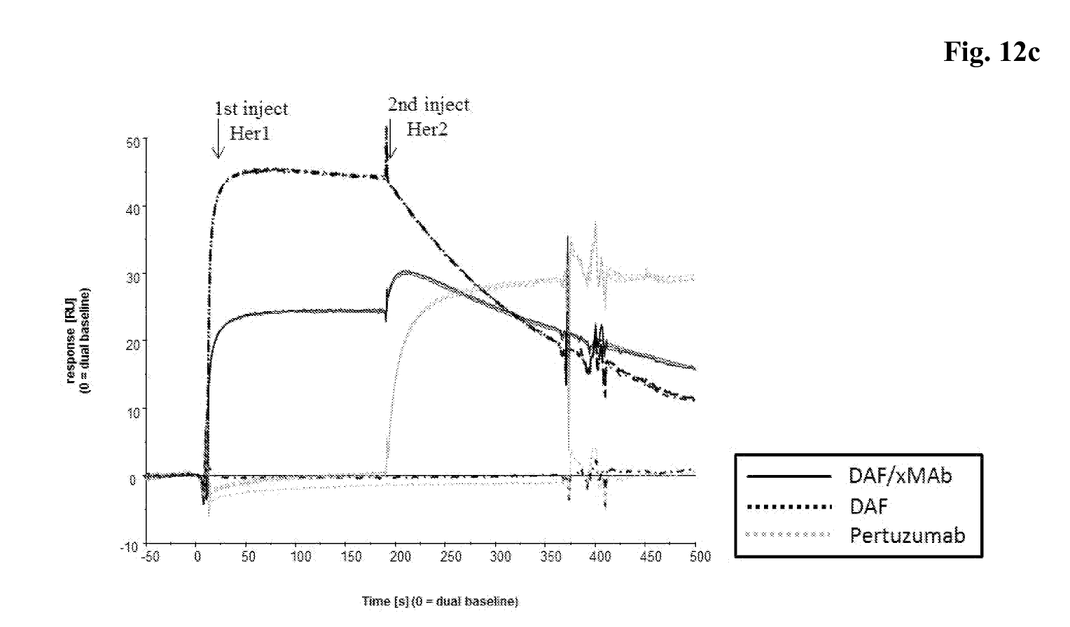

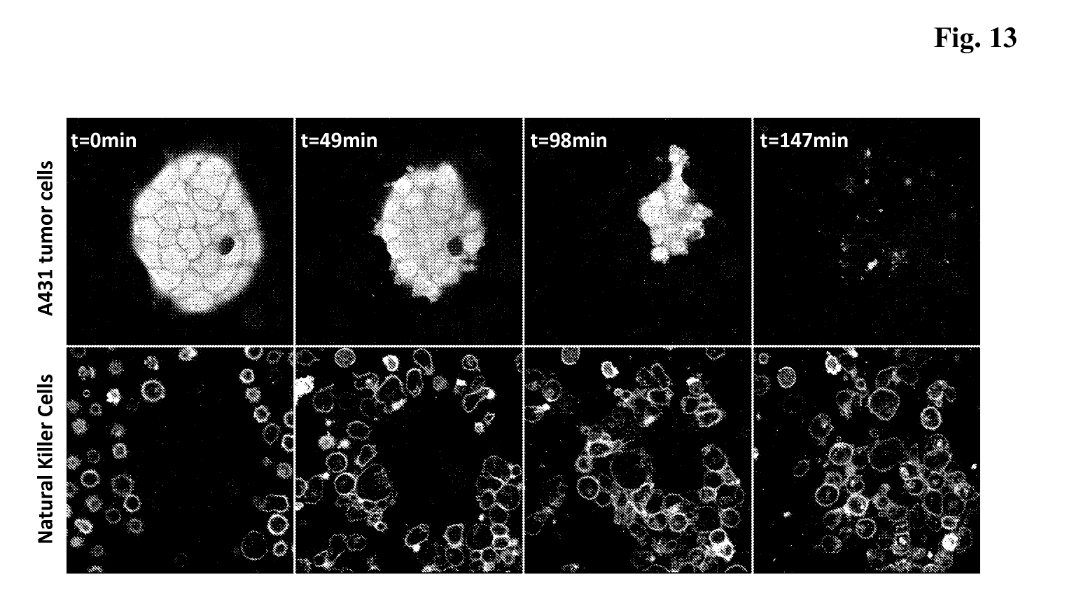

The term “full length antibody” denotes an antibody consisting of two antibody heavy chains and two antibody light chains (see FIG. 1). A heavy chain of full length antibody is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the heavy chain of full length antibody is a polypeptide consisting in N-terminal to C-terminal direction of VH, CH1, HR, CH2 and CH3. The light chain of full length antibody is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The full length antibody chains are linked together via inter-polypeptide disulfide bonds between the CL domain and the CH1 domain (i.e. between the light and heavy chain) and between the hinge regions of the full length antibody heavy chains. Examples of typical full length antibodies are natural antibodies like IgG (e.g. IgG 1 and IgG2), IgM, IgA, IgD, and IgE.) The full length antibodies according to the invention can be from a single species e.g. human, or they can be chimerized or humanized antibodies.

The full length antibodies according to the invention comprise two antigen binding sites each formed by a pair of VH and VL, which both specifically bind to a) either one single antigen or b) to bind to two different antigens (see below). The C-terminus of the heavy or light chain of said full length antibody denotes the last amino acid at the C-terminus of said heavy or light chain.

A full length antibody (or the light chain and heavy chain of a full length antibody) which specifically binds to two different antigens (e.g. a first antigen and second antigen, or a third and a fourth antigen) can e.g. obtained by diversifying the variable heavy chain and light chain domains VH and VL of a full length antibody so as to introduce dual specificities the techniques as described in WO 2008/027236; WO 2010/108127 and Bostrom, J., et al., Science 323 (2009) 1610-1614 (which are all incorporated by reference herein). The resulting VH and VL with dual specificities binding e.g. to a first antigen and second antigen can now be used in one arm of the multispecific according to the invention, while the other arm is specific for a third antigen (or a third and fourth antigen). The diversified VL and VH can bind the first epitope and second epitope simultaneously or mutually exclusively an can be selected e.g. from the group consisting of VEGF/HER2, VEGF-A/HER2, HER2/DR5, VEGF-A/PDGF, HER1/HER2, CD20/BR3, VEGF-A/VEGF-C, VEGF-C/VEGF-D, TNFalpha/TGF-beta, TNFalpha/IL-2, TNF alpha/IL-3, TNFalpha/IL-4, TNFalpha/IL-5, TNFalpha/IL6, TNFalpha/IL8, TNFalpha/IL-9, TNFalpha/IL-10, TNFalpha/IL-11, TNFalpha/IL-12, TNFalpha/IL-13, TNFalpha/IL-14, TNFalpha/IL-15, TNFalpha/IL-16, TNFalpha/IL-17, TNFalpha/IL-18, TNFalpha/IL-19, TNFalpha/IL-20, TNFalpha/IFNalpha, TNFalpha/CD4, VEGF/IL-8, VEGF/MET, VEGFR/MET receptor, HER2/Fc, HER2/HER3; HER1/HER2, HER1/HER3, EGFR/HER4, TNFalpha/IL-3, TNFalpha/IL-4, IL-13/CD40L, IL4/CD40L, TNFalpha/ICAM-1, TNFR1AL-IR, TNFR1/IL-6R, and TNFR1/IL-18R.

The terms “binding site” or “antigen-binding site” as used herein denotes the region(s) of an antibody molecule to which a ligand (e.g. the antigen or antigen fragment of it) actually binds and is derived from an antibody (or in case of a dual specific full length antibody the two ligands, e.g. the first and second antigen bind). The antigen-binding site includes antibody heavy chain variable domains (VH) pairs of VH/VL.

The antigen-binding sites that specifically bind to the desired antigen can be derived a) from known antibodies to the antigen or b) from new antibodies or antibody fragments obtained by de novo immunization methods using inter alia either the antigen protein or nucleic acid or fragments thereof or by phage display.

In case a dual specific antibody which binds to e.g. a first and second antigen, is desired, the VH and VL of the obtained antibody which binds to the first antigen have to modified/diversified as described in WO 2008/027236; WO 2010/108127 and Bostrom, J., et al., Science 323 (2009) 1610-1614 (which are all incorporated by reference herein).

An antigen-binding site of an antibody of the invention contains six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs).

Antibody specificity refers to selective recognition of the antibody for a particular epitope of an antigen. Natural antibodies, for example, are monospecific. Bispecific antibodies are antibodies which have two different antigen-binding specificities. Trispecific antibodies accordingly are antibodies to the invention which have three different antigen-binding specificities. Tetraspecific antibodies according to the invention are antibodies which have four different antigen-binding specificities.

Where an antibody has more than one specificity, the recognized epitopes may be associated with a single antigen or with more than one antigen.

The term “monospecific” antibody as used herein denotes an antibody that has one or more binding sites each of which bind to the same epitope of the same antigen.

The term “valent” as used within the current application denotes the presence of a specified number of binding sites in an antibody molecule. A natural antibody for example or a full length antibody according to the invention has two binding sites and is bivalent. In one embodiment the multispecific antibody according to the invention is bivalent. In one embodiment the multispecific antibody according to the invention is bivalent, trispecific or bivalent, tetraspecific.

The full length antibodies of the invention comprise immunoglobulin constant regions of one or more immunoglobulin classes. Immunoglobulin classes include IgG, IgM, IgA, IgD, and IgE isotypes and, in the case of IgG and IgA, their subtypes. In a preferred embodiment, an full length antibody of the invention has a constant domain structure of an IgG type antibody.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to Clq binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See, e.g., Morrison, S. L., et al., Proc. Natl. Acad. Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Other forms of “humanized antibodies” encompassed by the present invention are those in which the constant region has been additionally modified or changed from that of the original antibody to generate the properties according to the invention, especially in regard to Clq binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole, et al., Monoclonal Antibodies and Cancer Therapy, Alan R. Liss (1985) p. 77; and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to Clq binding and/or FcR binding, e.g. by “class switching” i.e. change or mutation of Fc parts (e.g. from IgG1 to IgG4 and/or IgG1/IgG4 mutation).

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable domain” (variable domain of a light chain (VL), variable domain of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain an antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. CDRs on each chain are separated by such framework amino acids. Especially, CDR3 of the heavy chain is the region which contributes most to antigen binding. CDR and FR regions are determined according to the standard definition of Kabat, E. A., et al., Sequences of Proteins of Immunological Interest, 5th ed., Public Health Service, National Institutes of Health Publication No. 91-3242, Bethesda, Md. (1991).

As used herein, the terms “binding”/“which specifically binds”/“specifically binding” refer to the binding of the antibody to an epitope of the antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). In one embodiment binding or specifically binding means a binding affinity (K_{D}) of 10⁻⁸ mol/l or less, preferably 10⁻⁹ M to 10⁻¹³ mol/l.

Thus, a multispecific antibody according to the invention preferably specifically binds to each antigen for which it is specific with a binding affinity (K_{D}) of 10⁻⁸ mol/l or less, preferably 10⁻⁹ to 10⁻¹³ mol/l.

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

In certain embodiments, an antibody is said to specifically bind an antigen when it preferentially recognizes its target antigen in a complex mixture of proteins and/or macromolecules.

In a further embodiment the multispecific antibody according to the invention is characterized in that said full length antibody is of human IgG1 subclass, or of human IgG1 subclass with the mutations L234A and L235A. In a further embodiment the multispecific antibody according to the invention is characterized in that said full length antibody is of human IgG2 subclass. In a further embodiment the multispecific antibody according to the invention is characterized in that said full length antibody is of human IgG3 subclass. In a further embodiment the multispecific antibody according to the invention is characterized in that said full length antibody is of human IgG4 subclass or, of human IgG4 subclass with the additional mutation S228P and L235E. In one embodiment the multispecific antibody according to the invention is characterized in that said full length antibody is of human IgG1 subclass, of human IgG4 subclass with the additional mutation S228P.

It has now been found that the multispecific antibodies according to the invention have improved characteristics such as biological or pharmacological activity, pharmacokinetic properties or toxicity. They can be used e.g. for the treatment of diseases such as cancer.

The term “constant region” as used within the current applications denotes the sum of the domains of an antibody other than the variable region. The constant region is not involved directly in binding of an antigen, but exhibit various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called α, δ, ε, γ, and μ, respectively. The light chain constant regions (CL) which can be found in all five antibody classes are called κ (kappa) and λ (lambda).

The term “constant region derived from human origin” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., (see e.g. Johnson, G. and Wu, T. T., Nucleic Acids Res. 28 (2000) 214-218; Kabat, E. A., et al., Proc. Natl. Acad. Sci. USA 72 (1975) 2785-2788).

While antibodies of the IgG4 subclass show reduced Fc receptor (FcγRIIIa) binding, antibodies of other IgG subclasses show strong binding. However Pro238, Asp265, Asp270, Asn297 (loss of Fc carbohydrate), Pro329, Leu234, Leu235, Gly236, Gly237, Ile253, Ser254, Lys288, Thr307, Gln311, Asn434, and His435 are residues which, if altered, provide also reduced Fc receptor binding (Shields, R. L., et al., J. Biol. Chem. 276 (2001) 6591-6604; Lund, J., et al., FASEB J. 9 (1995) 115-119; Morgan, A., et al., Immunology 86 (1995) 319-324; EP 0 307 434).

In one embodiment an antibody according to the invention has a reduced FcR binding compared to an IgG1 antibody. Thus the full length parent antibody is in regard to FcR binding of IgG4 subclass or of IgG1 or IgG2 subclass with a mutation in 5228, L234, L235 and/or D265, and/or contains the PVA236 mutation. In one embodiment the mutations in the full length parent antibody are S228P, L234A, L235A, L235E and/or PVA236. In another embodiment the mutations in the full length parent antibody are in IgG4 S228P and L235 E and in IgG1 L234A and L235A.

The constant region of an antibody is directly involved in ADCC (antibody-dependent cell-mediated cytotoxicity) and CDC (complement-dependent cytotoxicity). Complement activation (CDC) is initiated by binding of complement factor Clq to the constant region of most IgG antibody subclasses. Binding of Clq to an antibody is caused by defined protein-protein interactions at the so called binding site. Such constant region binding sites are known in the state of the art and described e.g. by Lukas, T. J., et al., J. Immunol. 127 (1981) 2555-2560; Bunkhouse, R. and Cobra, J. J., Mol. Immunol. 16 (1979) 907-917; Burton, D. R., et al., Nature 288 (1980) 338-344; Thomason, J. E., et al., Mol. Immunol. 37 (2000) 995-1004; Idiocies, E. E., et al., J. Immunol. 164 (2000) 4178-4184; Hearer, M., et al., J. Virol. 75 (2001) 12161-12168; Morgan, A., et al., Immunology 86 (1995) 319-324; and EP 0 307 434. Such constant region binding sites are, e.g., characterized by the amino acids L234, L235, D270, N297, E318, K320, K322, P331, and P329 (numbering according to EU index of Kabat).

The “EU numbering system” or “EU index (according to Kabat)” is generally used when referring to a residue or position in an immunoglobulin heavy chain constant region (e.g., the EU index is reported in Kabat, E. A., et al., Sequences of Proteins of Immunological Interest, 5th ed., Public Health Service, National Institutes of Health Publication No. 91-3242, Bethesda, Md. (1991) expressly incorporated herein by reference).

The term “antibody-dependent cellular cytotoxicity (ADCC)” refers to lysis of human target cells by an antibody according to the invention in the presence of effector cells. ADCC is measured preferably by the treatment of a preparation of antigen expressing cells with an antibody according to the invention in the presence of effector cells such as freshly isolated PBMC or purified effector cells from buffy coats, like monocytes or natural killer (NK) cells or a permanently growing NK cell line.

The term “complement-dependent cytotoxicity (CDC)” denotes a process initiated by binding of complement factor Clq to the Fc part of most IgG antibody subclasses. Binding of Clq to an antibody is caused by defined protein-protein interactions at the so called binding site. Such Fc part binding sites are known in the state of the art (see above). Such Fc part binding sites are, e.g., characterized by the amino acids L234, L235, D270, N297, E318, K320, K322, P331, and P329 (numbering according to EU index of Kabat). Antibodies of subclass IgG1, IgG2, and IgG3 usually show complement activation including Clq and C3 binding, whereas IgG4 does not activate the complement system and does not bind Clq and/or C3.

Cell-mediated effector functions of monoclonal antibodies can be enhanced by engineering their oligosaccharide component as described in Umana, P., et al., Nature Biotechnol. 17 (1999) 176-180, and U.S. Pat. No. 6,602,684. IgG1 type antibodies, the most commonly used therapeutic antibodies, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M., R., et al., Glycobiology 5 (1995) 813-822; Jefferis, R., et al., Immunol. Rev. 163 (1998) 59-76; Wright, A., and Morrison, S. L., Trends Biotechnol. 15 (1997) 26-32). Umana, P., et al., Nature Biotechnol. 17 (1999) 176-180 and WO 99/54342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the Asn297 carbohydrate or its elimination affect also binding to FcγR and Clq (Umana, P., et al., Nature Biotechnol. 17 (1999) 176-180; Davies, J., et al., Biotechnol. Bioeng. 74 (2001) 288-294; Mimura, Y., et al., J. Biol. Chem. 276 (2001) 45539-45547; Radaev, S., et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Methods to enhance cell-mediated effector functions of monoclonal antibodies are reported e.g. in WO 2005/018572, WO 2006/116260, WO 2006/114700, WO 2004/065540, WO 2005/011735, WO 2005/027966, WO 1997/028267, US 2006/0134709, US 2005/0054048, US 2005/0152894, WO 2003/035835, WO 2000/061739.

In one preferred embodiment of the invention, the tri- or tetraspecific antibody is glycosylated (if it comprises an Fc part of IgG1, IgG2, IgG3 or IgG4 subclass, preferably of IgG1 or IgG3 subclass) with a sugar chain at Asn297 whereby the amount of fucose within said sugar chain is 65% or lower (Numbering according to Kabat). In another embodiment is the amount of fucose within said sugar chain is between 5% and 65%, preferably between 20% and 40%. In another embodiment is the amount of fucose within said sugar chain is between 0%. “Asn297” according to the invention means amino acid asparagine located at about position 297 in the Fc region. Based on minor sequence variations of antibodies, Asn297 can also be located some amino acids (usually not more than ±3 amino acids) upstream or downstream of position 297, i.e. between position 294 and 300. In one embodiment the glycosylated antibody according to the invention the IgG subclass is of human IgG1 subclass, of human IgG1 subclass with the mutations L234A and L235A or of IgG3 subclass. In a further embodiment the amount of N-glycolylneuraminic acid (NGNA) is 1% or less and/or the amount of N-terminal alpha-1,3-galactose is 1% or less within said sugar chain. The sugar chain show preferably the characteristics of N-linked glycans attached to Asn297 of an antibody recombinantly expressed in a CHO cell.

The term “the sugar chains show characteristics of N-linked glycans attached to Asn297 of an antibody recombinantly expressed in a CHO cell” denotes that the sugar chain at Asn297 of the full length parent antibody according to the invention has the same structure and sugar residue sequence except for the fucose residue as those of the same antibody expressed in unmodified CHO cells, e.g. as those reported in WO 2006/103100.

The term “NGNA” as used within this application denotes the sugar residue N-glycolylneuraminic acid.

Glycosylation of human IgG1 or IgG3 occurs at Asn297 as core fucosylated biantennary complex oligosaccharide glycosylation terminated with up to two Gal residues. Human constant heavy chain regions of the IgG1 or IgG3 subclass are reported in detail by Kabat, E. A., et al., Sequences of Proteins of Immunological Interest, 5th ed., Public Health Service, National Institutes of Health Publication No. 91-3242, Bethesda, Md. (1991), and by Brüggemann, M., et al., J. Exp. Med. 166 (1987) 1351-1361; Love, T. W., et al., Methods Enzymol. 178 (1989) 515-527. These structures are designated as G0, G1 (α-1,6- or α-1,3-), or G2 glycan residues, depending from the amount of terminal Gal residues (Raju, T. S., Bioprocess Int. 1 (2003) 44-53). CHO type glycosylation of antibody Fc parts is e.g. described by Routier, F. H., Glycoconjugate J. 14 (1997) 201-207. Antibodies which are recombinantly expressed in non-glycomodified CHO host cells usually are fucosylated at Asn297 in an amount of at least 85%. The modified oligosaccharides of the full length parent antibody may be hybrid or complex. Preferably the bisected, reduced/not-fucosylated oligosaccharides are hybrid. In another embodiment, the bisected, reduced/not-fucosylated oligosaccharides are complex.

According to the invention “amount of fucose” means the amount of said sugar within the sugar chain at Asn297, related to the sum of all glycostructures attached to Asn297 (e.g. complex, hybrid and high mannose structures) measured by MALDI-TOF mass spectrometry and calculated as average value. The relative amount of fucose is the percentage of fucose-containing structures related to all glycostructures identified in an N-Glycosidase F treated sample (e.g. complex, hybrid and oligo- and high-mannose structures, resp.) by MALDI-TOF.

The antibody according to the invention is produced by recombinant means. Thus, one aspect of the current invention is a nucleic acid encoding the antibody according to the invention and a further aspect is a cell comprising said nucleic acid encoding an antibody according to the invention. Methods for recombinant production are widely known in the state of the art and comprise protein expression in prokaryotic and eukaryotic cells with subsequent isolation of the antibody and usually purification to a pharmaceutically acceptable purity. For the expression of the antibodies as aforementioned in a host cell, nucleic acids encoding the respective modified light and heavy chains are inserted into expression vectors by standard methods. Expression is performed in appropriate prokaryotic or eukaryotic host cells like CHO cells, NS0 cells, SP2/0 cells, HEK293 cells, COS cells, PER.C6 cells, yeast, or E. coli cells, and the antibody is recovered from the cells (supernatant or cells after lysis). General methods for recombinant production of antibodies are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The tri- or tetraspecific antibodies according to the invention are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies is readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

Amino acid sequence variants (or mutants) of the tri- or tetraspecific antibody are prepared by introducing appropriate nucleotide changes into the antibody DNA, or by nucleotide synthesis. Such modifications can be performed, however, only in a very limited range, e.g. as described above. For example, the modifications do not alter the above mentioned antibody characteristics such as the IgG isotype and antigen binding, but may improve the yield of the recombinant production, protein stability or facilitate the purification.

The term “host cell” as used in the current application denotes any kind of cellular system which can be engineered to generate the antibodies according to the current invention. In one embodiment HEK293 cells and CHO cells are used as host cells. As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.-J., J. Immunol. Methods 194 (1996) 191-199.

The control sequences that are suitable for prokaryotes, for example, include a promoter, optionally an operator sequence, and a ribosome binding site. Eukaryotic cells are known to utilize promoters, enhancers and polyadenylation signals.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

Purification of antibodies is performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., (ed.), Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

One aspect of the invention is a pharmaceutical composition comprising an antibody according to the invention. Another aspect of the invention is the use of an antibody according to the invention for the manufacture of a pharmaceutical composition. A further aspect of the invention is a method for the manufacture of a pharmaceutical composition comprising an antibody according to the invention. In another aspect, the present invention provides a composition, e.g. a pharmaceutical composition, containing an antibody according to the present invention, formulated together with a pharmaceutical carrier.

One embodiment of the invention is the multispecific antibody according to the invention for the treatment of cancer.

Another aspect of the invention is said pharmaceutical composition for the treatment of cancer.

Another aspect of the invention is the use of an antibody according to the invention for the manufacture of a medicament for the treatment of cancer.

Another aspect of the invention is method of treatment of patient suffering from cancer by administering an antibody according to the invention to a patient in the need of such treatment.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. As will be appreciated by the skilled artisan, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, sub arachnoid, intraspinal, epidural and intrasternal injection and infusion.

The term cancer as used herein refers to proliferative diseases, such as lymphomas, lymphocytic leukemias, lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, the carrier preferably is an isotonic buffered saline solution.

Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

The term “transformation” as used herein refers to process of transfer of a vectors/nucleic acid into a host cell. If cells without formidable cell wall barriers are used as host cells, transfection is carried out e.g. by the calcium phosphate precipitation method as described by Graham, and Van der Eh, Virology 52 (1978) 546ff. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride as described by Cohen, F. N, et al., PNAS 69 (1972) 7110 et seq.

As used herein, “expression” refers to the process by which a nucleic acid is transcribed into mRNA and/or to the process by which the transcribed mRNA (also referred to as transcript) is subsequently being translated into peptides, polypeptides, or proteins. The transcripts and the encoded polypeptides are collectively referred to as gene product. If the polynucleotide is derived from genomic DNA, expression in a eukaryotic cell may include splicing of the mRNA.

A “vector” is a nucleic acid molecule, in particular self-replicating, which transfers an inserted nucleic acid molecule into and/or between host cells. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a polynucleotide which, when introduced into an appropriate host cell, can be transcribed and translated into a polypeptide. An “expression system” usually refers to a suitable host cell comprised of an expression vector that can function to yield a desired expression product.

Still a further aspect of the invention is a multispecific antibody, comprising:
A) a) the light chain and heavy chain of a full length antibody which specifically binds to a first antigen and second antigen; and
   b) the light chain and heavy chain of a full length antibody which specifically binds to a third antigen, wherein the N-terminus of the heavy chain is connected to the C-terminus of the light chain via a peptide linker;
or
B) a) the light chain and heavy chain of a full length antibody which specifically binds to a first antigen; and
   b) the light chain and heavy chain of a full length antibody which specifically binds to a second antigen and third antigen, wherein the N-terminus of the heavy chain is connected to the C-terminus of the light chain via a peptide linker;
or
C) a) the light chain and heavy chain of a full length antibody which specifically binds to a first antigen and a second antigen; and
   b) the light chain and heavy chain of a full length antibody which specifically binds to a third antigen and fourth antigen, wherein the N-terminus of the heavy chain is connected to the C-terminus of the light chain via a peptide linker.

The term “peptide linker” as used within the invention denotes a peptide with amino acid sequences, which is preferably of synthetic origin. These peptides according to invention are used to connect the C-terminus of the light chain to the N-terminus of heavy chain of the second full length antibody (that specifically binds to a second antigen) via a peptide linker. The peptide linker within the second full length antibody heavy and light chain is a peptide with an amino acid sequence with a length of at least 30 amino acids, preferably with a length of 32 to 50 amino acids. In one the peptide linker is a peptide with an amino acid sequence with a length of 32 to 40 amino acids. In one embodiment said linker is (G×S)n with G=glycine, S=serine, (x=3, n=8, 9 or 10 and m=0, 1, 2 or 3) or (x=4 and n=6, 7 or 8 and m=0, 1, 2 or 3), preferably with x=4, n=6 or 7 and m=0, 1, 2 or 3, more preferably with x=4, n=7 and m=2. In one embodiment said linker is (G4S)6G2.

One embodiment of such multispecific antibodies is give in the Examples Table 1: Trispecific Her1/Her3-scFab-IGF1R comprising the amino acid sequences of SEQ ID NOs: 4, 11 and 13.

The following examples, sequence listing and figures are provided to aid the understanding of the present invention, the true scope of which is set forth in the appended claims. It is understood that modifications can be made in the procedures set forth without departing from the spirit of the invention.

### Description of the Amino Acid Sequences

### SEQ ID NO: 1: HC/knob/Her2/VEGF

### SEQ ID NO: 2: HC/hole/Her2/VEGF

### SEQ ID NO: 3: HC/knob/Her1/Her3

### SEQ ID NO: 4: HC/hole/Her1/Her3

### SEQ ID NO: 5: HC/hole/xAng2

### SEQ ID NO: 6: HC/knob/xAng2

### SEQ ID NO: 7: HC/hole/xIGF1R

### SEQ ID NO: 8: HC/hole/xHer3

### SEQ ID NO: 9: HC/hole/xHer2

### SEQ ID NO: 10: HC/hole/xcMet

### SEQ ID NO: 11: HC/hole/scFabIGF1R

### SEQ ID NO: 12: HC/hole/xHer1/Her3

### SEQ ID NO: 13: LC/Her1/Her3

### SEQ ID NO: 14: LC/Her2/VEGF

### SEQ ID NO: 15: LC/xAng2

### SEQ ID NO: 16: LC/xIGF1R

### SEQ ID NO: 17: LC/xHer3

### SEQ ID NO: 18: LC/xHer2

### SEQ ID NO: 19: LC/xcMet

### SEQ ID NO: 20: LC/xHer1/Her3

In the following, embodiments of the invention are listed:
1. A multispecific antibody, comprising:
   A) a) the light chain and heavy chain of a full length antibody which specifically binds to a first antigen and second antigen; and
      b) the modified light chain and modified heavy chain of a full length antibody which specifically binds to a third antigen, wherein the variable domains VL and VH are replaced by each other, and/or wherein the constant domains CL and CH1 are replaced by each other;
   or
   B) a) the light chain and heavy chain of a full length antibody which specifically binds to a first antigen; and
      b) the modified light chain and modified heavy chain of a full length antibody which specifically binds to a second antigen and third antigen, wherein the variable domains VL and VH are replaced by each other, and/or wherein the constant domains CL and CH1 are replaced by each other;
   or
   C) a) the light chain and heavy chain of a full length antibody which specifically binds to a first antigen and a second antigen; and
      b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
2. The multispecific antibody according to embodiment 1, characterized in that the antibody is a bivalent, tri- or tetraspecific antibody.
3. The multispecific antibody according to embodiment 1, characterized in that the antibody is trispecific and comprises
   A) a) the light chain and heavy chain of a full length antibody which specifically binds to a first antigen and second antigen; and
      b) the modified light chain and modified heavy chain of a full length antibody which specifically binds to a third antigen, wherein the variable domains VL and VH are replaced by each other, and/or wherein the constant domains CL and CH1 are replaced by each other;
   or
   B) a) the light chain and heavy chain of a full length antibody which specifically binds to a first antigen; and
      b) the modified light chain and modified heavy chain of a full length antibody which specifically binds to a second antigen and third antigen, wherein the variable domains VL and VH are replaced by each other, and/or wherein the constant domains CL and CH1 are replaced by each other.
4. The multispecific antibody according to embodiment 3, wherein
   under A) the first antigen is human HER1, the second antigen human HER3 and the third antigen is human HER2; or
   under B) the first antigen is human HER2, the second antigen human HER1 and the third antigen is human HER3.
5. The multispecific antibody according to embodiment 1, wherein the antibody is a bivalent, trispecific antibody and comprises a
   a) the light chain and heavy chain of a full length antibody which specifically binds to human HER1 and human HER3; and
   b) the modified light chain and modified heavy chain of a full length antibody which specifically binds to human HER2, wherein the variable domains VL and VH are replaced by each other, and/or wherein the constant domains CL and CH1 are replaced by each other;
6. The multispecific antibody according to embodiment 5, wherein the antibody is characterized in comprising the amino acid sequences of SEQ ID NOs: 4, 9, 13 and 18
7. The multispecific antibody according to embodiment 3, wherein
   under A) the first antigen is human HER1, the second antigen human HER3 and the third antigen is human cMET; or
   under B) the first antigen is human cMET, the second antigen human HER1 and the third antigen is human HER3.
8. The multispecific antibody according to embodiment 1, characterized in that the antibody is tetraspecific and comprises
   a) the light chain and heavy chain of a full length antibody which specifically binds to a first antigen and a second antigen; and
   b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
9. The multispecific antibody according to any one of embodiments 1 to 5, 7 and 8, wherein in the modified light chain and modified heavy chain under b) the variable domains VL and VH are replaced by each other; and wherein the constant domains CL and CH1 are replaced by each other.
10. The multispecific antibody according to any one of embodiments claims 1 to 5, 7 and 8, wherein in the modified light chain and modified heavy chain under b) (only) the variable domains VL and VH are replaced by each other,
11. The multispecific antibody according to any one of embodiments 1 to 5, 7 and 8, wherein in the modified light chain and modified heavy chain under b) (only) the constant domains CL and CH1 are replaced by each other.
12. The multispecific antibody according to any one of embodiments 1 to 11, characterized in that
   the CH3 domain of the heavy chain of the full length antibody of a) and
   the CH3 domain of the modified heavy chain of the full length antibody of b) each meet at an interface which comprises an original interface
   between the antibody CH3 domains;
      wherein said interface is altered to promote the formation of the trispecific or tetraspecific antibody, wherein the alteration is characterized in that:
      i) the CH3 domain of one heavy chain is altered,
      so that within the original interface the CH3 domain of one heavy chain that meets the original interface of the CH3 domain of the other heavy chain within the tri- or tetraspecific antibody,
      an amino acid residue is replaced with an amino acid residue having a larger side chain volume, thereby generating a protuberance within the interface of the CH3 domain of one heavy chain which is positionable in a cavity within the interface of the CH3 domain of the other heavy chain
      and
      ii) the CH3 domain of the other heavy chain is altered, so that within the original interface of the second CH3 domain that meets the original interface of the first CH3 domain within the tri- or tetraspecific antibody
      an amino acid residue is replaced with an amino acid residue having a smaller side chain volume, thereby generating a cavity within the interface of the second CH3 domain within which a protuberance within the interface of the first CH3 domain is positionable.
13. The multispecific antibody according to embodiment 12, characterized in that
   the said amino acid residue having a larger side chain volume is selected from the group consisting of arginine (R), phenylalanine (F), tyrosine (Y), tryptophan (W) and said amino acid residue having a smaller side chain volume is selected from the group consisting of alanine (A), serine (S), threonine (T), valine (V).
14. The multispecific antibody according to embodiments 12 or 13, characterized in that
   both CH3 domains are further altered by the introduction of cysteine (C) as amino acid in the corresponding positions of each CH3 domain such that a disulfide bridge between both CH3 domains can be formed.
15. A method for the preparation of a multispecific antibody according to embodiments 1 to 14
   comprising the steps of
   a) transforming a host cell with vectors comprising nucleic acid molecules encoding
      A) a) the light chain and heavy chain of a full length antibody which specifically binds to a first antigen and second antigen; and
         b) the modified light chain and modified heavy chain of a full length antibody which specifically binds to a third antigen, wherein the variable domains VL and VH are replaced by each other, and/or wherein the constant domains CL and CH1 are replaced by each other;
      or
      B) a) the light chain and heavy chain of a full length antibody which specifically binds to a first antigen; and
         b) the modified light chain and modified heavy chain of a full length antibody which specifically binds to a second antigen and third antigen, wherein the variable domains VL and VH are replaced by each other, and/or wherein the constant domains CL and CH1 are replaced by each other;
      or
      C) a) the light chain and heavy chain of a full length antibody which specifically binds to a first antigen and a second
         b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
   b) culturing the host cell under conditions that allow synthesis of said antibody molecule; and
   c) recovering said antibody molecule from said culture.
16. Nucleic acid encoding the multispecific antigen binding protein according to embodiments 1 to 14.
17. Vectors comprising nucleic acid encoding the multispecific antigen binding protein according to embodiments 1 to 14.
18. A host cell comprising the vectors according to embodiment 17.
19. A composition, preferably a pharmaceutical or a diagnostic composition of the antibody according to embodiments 1 to 14.
20. A pharmaceutical composition comprising an antibody according to embodiments 1 to 14 and at least one pharmaceutically acceptable excipient.
21. An antibody according to any one of embodiments 1 to 14 for use in the treatment of cancer.
22. Use of an antibody according to any one of embodiments 1 to 14 for the manufacture of a medicament for the treatment of cancer.
23. A method for the treatment of a patient in need of therapy, characterized by administering to the patient a therapeutically effective amount of an antibody to embodiments 1 to 14.
24. A method for the treatment of a patient suffering from cancer, characterized by administering to the patient a therapeutically effective amount of an antibody to embodiments 1 to 14

### EXAMPLES

### Materials & Methods

### Recombinant DNA Techniques

Standard methods were used to manipulate DNA as described in Sambrook, J., et al., Molecular Cloning: A Laboratory Manual; Cold Spring Harbor Laboratory Press, Cold Spring Harbor, N.Y. (1989). The molecular biological reagents were used according to the manufacturer's instructions.

### DNA and Protein Sequence Analysis and Sequence Data Management

General information regarding the nucleotide sequences of human immunoglobulins light and heavy chains is given in: Kabat, E. A., et al., Sequences of Proteins of Immunological Interest, 5th ed., Public Health Service, National Institutes of Health Publication No 91-3242, Bethesda (1991). Amino acids of antibody chains are numbered according to EU numbering (Edelman, G. M., et al., PNAS 63 (1969) 78-85; Kabat, E. A., et al., Sequences of Proteins of Immunological Interest, 5th ed., NIH Publication No 91-3242 (1991)). The GCG's (Genetics Computer Group, Madison, Wis.) software package version 10.2 and Infomax's Vector NTT Advance suite version 8.0 was used for sequence creation, mapping, analysis, annotation and illustration.

### DNA Sequencing

DNA sequences were determined by double strand sequencing performed at SequiServe (Vaterstetten, Germany) and Geneart AG (Regensburg, Germany).

### Gene Synthesis

Desired gene segments were prepared by Geneart AG (Regensburg, Germany) from synthetic oligonucleotides and PCR products by automated gene synthesis. The gene segments which are flanked by singular restriction endonuclease cleavage sites were cloned into pGA18 (ampR) plasmids. The plasmid DNA was purified from transformed bacteria and concentration determined by UV spectroscopy. The DNA sequence of the subcloned gene fragments was confirmed by DNA sequencing.

### Construction of the Expression Plasmids

A Roche expression vector was used for the construction of all heavy and light chain encoding expression plasmids. The vector is composed of the following elements:
a hygromycin resistance gene as a selection marker,
an origin of replication, oriP, of Epstein-Barr virus (EBV),
an origin of replication from the vector pUC 18 which allows replication of this plasmid in E. coli
a beta-lactamase gene which confers ampicillin resistance in E. coli,
the immediate early enhancer and promoter from the human cytomegalovirus (HCMV),
the human 1-immunoglobulin polyadenylation (“poly A”) signal sequence, and

The immunoglobulin genes comprising the heavy or light chain as well as crossmab constructs with CH-CL crossover were prepared by gene synthesis and cloned into pGA18 (ampR) plasmids as described. Variable heavy chain constructs were constructed by directional cloning using a 5′ BamHI upstream of the cds and 3′ KpnI restriction site located in the CH1 domain. Variable light chain constructs were ordered as gene synthesis comprising VL and CL and constructed by directional cloning using a 5′ BamHI upstream of the cds and 3′ XbaI restriction site located downstream of the stop codon. Crossmab antibodies were constructed either by gene synthesis of full coding sequence (VL-CH1 or VH-CL-CH2-CH3) or as partial gene synthesis with unique restriction sites in the coding sequence. In the case of the crossed light chain (VL-CH1) only gene synthesis covering the whole cds with 5′ BamHI and 3′ XbaI restriction sites were ordered. For heavy chain constructs also a unique 3′ XhoI restriction site in the CH2 domain of the heavy chain vector was used for directional cloning with a 5′ BamHI restriction site. The final expression vectors were transformed into E. coli cells, expression plasmid DNA was isolated (Miniprep) and subjected to restriction enzyme analysis and DNA sequencing. Correct clones were grown in 150 ml LB-Amp medium, again plasmid DNA was isolated (Maxiprep) and sequence integrity confirmed by DNA sequencing.

### Transient Expression of Immunoglobulin Variants in HEK293 Cells

Recombinant immunoglobulin variants were expressed by transient transfection of human embryonic kidney 293-F cells using the FreeStyle™ 293 Expression System according to the manufacturer's instruction (Invitrogen, USA). For small scale test expressions 30 ml of 0.5×10⁶ HEK293F cells/ml were seeded one day prior to transfection. The next day, plasmid DNA (1 μg DNA per ml culture volume) was mixed with 1.2 ml Opti-MEMO I Reduced Serum Medium (Invitrogen, Carlsbad, Calif., USA) followed by addition of 40 μl of 293Fectin™ Transfection Reagent (Invitrogen, Carlsbad, Calif., USA). The mixture was incubated for 15 min at room temperature and added drop wise to the cells. One day post-transfection each flask was fed with 300 μl L-Glutamine (200 mM, Sigma-Aldrich, Steinheim, Germany) and 600 μl feed7 containing L-asparagine, HyPep 1510, ammonium-Fe(III) citrate, ethanolamine, trace elements, D-glucose, FreeStyle medium without RDMI. Three days post-transfection cell concentration, viability and glucose concentration in the medium were determined using an automated cell viability analyzer (Vi-CELL™ XR, Beckman Coulter, Fullerton, Calif., USA) and a glucose meter (Accu-CHEK® Sensor comfort, Roche Diagnostics GmbH, Mannheim, Germany). In addition each flask was fed with 300 μl of L-glutamine, 300 μl non-essential amino acids solution (PAN™ Biotech, Aidenbach, Germany), 300 μl sodium pyruvate (100 mM, Gibco, Invitrogen), 1.2 ml feed7 and ad 5 g/L glucose (D-(+)-Glucose solution 45%, Sigma). Finally, six days post-transfection antibodies were harvested by centrifugation at 3500 rpm in a X3R Multifuge (Heraeus, Buckinghamshire, England) for 15 min at ambient temperature, the supernatant was sterile filtered through a Steriflip filter unit (0.22 mm Millipore Express PLUS PES membrane, Millipore, Bedford, Mass.) and stored at −20° C. until further use

### Purification of Bispecific and Control Antibodies

Bivalent trispecific or tetraspecific and control antibodies were purified from cell culture supernatants by affinity chromatography using Protein A-Sepharose™ (GE Healthcare, Sweden) and Superdex200 size exclusion chromatography. Briefly, sterile filtered cell culture supernatants were applied on a HiTrap ProteinA HP (5 ml) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. Antibody and antibody variants were eluted with 0.1 M citrate buffer, pH 2.8, and the protein containing fractions were neutralized with 0.1 ml 1 M Tris, pH 8.5. Eluted protein fractions were pooled, concentrated with an Amicon Ultra centrifugal filter device (MWCO: 30 K, Millipore) to a volume of 3 ml and loaded on a Superdex200 HiLoad 120 ml 16/60 gel filtration column (GE Healthcare, Sweden) equilibrated with 20 mM Histidin, 140 mM NaCl, pH 6.0. Fractions containing purified bispecific and control antibodies with less than 5% high molecular weight aggregates were pooled and stored as 1.0 mg/ml aliquots at -80° C.

### Protein Quantification

Proteins were quantified by affinity chromatography using the automated Ultimate 3000 system (Dionex, Idstein, Germany) with a pre-packed Poros® A protein A column (Applied Biosystems, Foster City, Calif., USA). All samples were loaded in buffer A (0.2 M Na₂HPO₄.[2H₂O], pH 7.4) and eluted in buffer B (0.1 M citric acid, 0.2 M NaCl, pH 2.5). In order to determine the protein concentration an extinction coefficient of 1.62 was used for all samples.

### Analysis of Purified Proteins

The protein concentration of purified protein samples was determined by measuring the optical density (OD) at 280 nm, using the molar extinction coefficient calculated on the basis of the amino acid sequence. Purity and molecular weight of bispecific and control antibodies were analyzed by SDS-PAGE in the presence and absence of a reducing agent (5 mM 1,4-dithiothreitol) and staining with Coomassie brilliant blue. The NuPAGE® Pre-Cast gel system (Invitrogcn, USA) was used according to the manufacturer's instruction (4-20% Tris-Glycine gels). The aggregate content of bispecific and control antibody samples was analyzed by high-performance SEC using a Superdex 200 analytical size-exclusion column (GE Healthcare, Sweden) in 200 mM KH₂PO₄, 250 mM KCl, pH 7.0 running buffer at 25° C. 25 μg protein were injected on the column at a flow rate of 0.5 ml/min and eluted isocratic over 50 minutes. Integrity of the amino acid backbone of reduced bispecific antibody light and heavy chains was verified by NanoElectrospray Q-TOF mass spectrometry after removal of N-glycans by enzymatic treatment with Peptide-N-Glycosidase F (Roche Molecular Biochemicals).

### Immunoprecipitation for Small Scale Analysis

30 μg of protein were diluted in PBS supplemented with 5% Tween®20 (PBS-T, pH 7.4, Fluka Analytical, Steinheim, Germany) to an equal total reaction volume for all samples. 126 μl Dynabeads® Protein A (0.24 μg human IgG per μl Dynabeads binding capacity, Invitrogen, Carlsbad, Calif., USA) were added and the solution was incubated for 90 to 120 min at room temperature and 20 rpm to allow binding of the human IgG Fc to protein A linked to magnetic beads (1.4 ml total reaction volume). Beads were washed three times with 1 ml PBS-T, centrifuged for 30 seconds at 0.4×g to collect the solution at the bottom of the tube. Supernatant was discarded and Dynabeads were incubated with 30 μl of 100 mM citrate, pH 3 (Citric acid monohydrate, Sigma) to elute the proteins. Afterwards the solution was neutralized with 3 μl of 2M Tris, pH 9 (Fisher Scientific).

### Analytical HPLC

Antibodies were analyzed using a Agilent HPLC 1100 (Agilent Technologies, Paulo Alto, Calif., USA) with a TSK-GEL G3000SW gel filtration column (7.5 mm ID×30 cm, TosoHaas Corp., Montgomeryville, Pa., USA). 18 μl of the eluted proteins were loaded onto the column in Buffer A (0.05 M K₂HPO₄/KH₂PO₄ in 300 mM NaCl, pH 7.5) and separated based on size.

### Reducing and Non-Reducing SDS-Page

7 μl of the eluted proteins were mixed with 2× sample buffer (NuPAGE® LDS Sample buffer, Invitrogen, Carlsbad, Calif., USA) and another 7 μl were mixed with 2× sample buffer containing 10% reducing agent (NuPAGE® Sample Reducing Agent, Invitrogen, Carlsbad, Calif., USA). Samples were heated to 70° for 10 min and loaded onto a pre-cast NuPAGE® 4-12% BisTris Gel (Invitrogen, Carlsbad, Calif., USA). The gel was run for 45 min at 200V and 125 mA. Afterwards the gel was washed three times with Millipore water and stained with SimplyBlue™ SafeStain (Invitrogen, Carlsbad, Calif., USA). The gel was destained overnight in Millipore water.

### Cell Lines

A431 were maintained in RPMI 1640 medium (Gibco), supplemented with 4 mM L-glutamine, 0.1 mM non-essential amino acids and 10% heat inactivated fetal calf serum (Gibco). MDA-MB 175 VII cells were maintained in DMEM/F12 medium (Gibco) supplemented with GlutaMax. Propagation of cell lines followed standard cell culture protocols.

### Surface Plasmon Resonance

All experiments were performed on a Biacore T100 (GE Healthcare). Experimental results were analyzed using the T100 control and evaluation software package (GE Healthcare, v2.03). The assay format was a ‘multi cycle kinetic’ measurement on a CM5-chip. Antibody to be analyzed was captured via amine coupled anti-human IgG-Fc antibody (GE Healthcare BR-1008-39). DAF and pertuzumab were used as reference controls. Using a concentration series, seven increasing concentrations of each of the antigens (human Her1, Her2, and Her3 ectodomain) were injected separately. Kinetic characterization of HerX binding to respective MAbs<HerX> at 37° C.: Standard kinetics were evaluated by fitting of the observed time course of surface plasmon resonance signals for the association and dissociation phase with a Langmuir 1:1 binding model with double referencing (against c=0 nM and FC 1=blank surface) by Biacore evaluation software. Running buffer was PBS-T. Dilution buffer was PBST containing BSA (c=1 mg/mL). Capturing of MAbs<HerX> on flow cell 2, 3, and 4 with a concentration of approx. c=1 nM, flow 50/min, time 72 sec. Analyte sample: Seven increasing concentrations of HerX at a flow rate of 500/min were injected for 180 sec association time (c=1.23-900 nM, dilution factor 3). Dissociation time: 1800 sec. Each concentration was analyzed as duplicate. Final regeneration was performed after each cycle using 3 M MgCl2 (recommended by vendor) with a contact time of 120 sec and a flow rate of 50 μl/min. Analysis of simultaneous binding: Her2/Her3, Her3/Her2 or Her1/Her2 were injected consecutively using the dual inject mode with a contact time of 180 sec. each. The antigen concentration was chosen for each antigen at the saturation as observed in the kinetics experiment. As control a 2nd inject of the identical antigen did not raise response level, demonstrating that equilibrium was reached. A temperature of 25° C. was chosen to minimize dissociation. Triplicates for each combination were determined. Flow rate 30 mL/min, dual inject with two injects, each 180 sec.

Various multispecific antibodies according to the invention were designed to evaluate the concept (see Table 1 below). Typically they include knobs-into-holes modification in the CH3 domain (as can be seen in the respective sequences)

TABLE 1
Design of multispecific antibodies according to the invention: Numbers
indicate sequence numbers as in sequence listing (x indicates that in
the light and heavy chain the CH1 and the CL have been exchanged).

| | Bispecific | Trispecific | Trispecific | Trispecific | Trispecific | Tetraspecific |
|---|---|---|---|---|---|---|
| | Her2/Vegf | Her2/Vegf | Her2/Vegf | Her2/Vegf | Her2/Vegf | Her2/Vegf |
| # | (with KiH) | xAng2(hole) | xAng2(knob) | xIGF1R | xHer3 | xHer1/3 |
| HC1 | 1 | 1 | 2 | 1 | 1 | 1 |
| HC2 | 2 | 5 | 6 | 7 | 8 | 12 |
| LC1 | 14 | 14 | 14 | 14 | 14 | 14 |
| LC2 | — | 15 | 15 | 16 | 17 | 20 |
| | Bispecific | | | | | Trispecific |
| | parent | Trispecific | Trispecific | Trispecific | Trispecific | Her1/Her3 |
| | Her1/Her3 | Her1/Her3 | Her1/Her3 | Her1/Her3 | Her1/Her3 | scFab- |
| # | (with KiH) | xcMet | xHer2 | xAng2(hole) | xAng2(knob) | IGF1R |
| HC1 | 3 | 4 | 4 | 3 | 4 | 4 |
| HC2 | 4 | 10 | 9 | 5 | 2 | 11 |
| LC1 | 13 | 13 | 13 | 13 | 13 | 13 |
| LC2 | — | 19 | 18 | 15 | 15 | — |

### EXAMPLES

### Example 1

### Analysis of Knobs-into-Holes VEGF-Her2 DAF (Dual Affinity Antibody)

The VEGF-Her2-DAF has been described previously (Bostrom, J., et al., Science 323 (2009) 1610-1614). To provide evidence that the knobs-into-holes technology does not interfere with the expression of the VEGF-Her2-DAF we engineered the “knobs-into-holes” (KiH) amino acid exchanges in the heavy chain of this antibody (heavy chain 1: T366W; heavy chain 2: T366S, L368A, Y407V). Additionally, a disulfide bridge was introduced in the CH3 domain of this antibody (heavy chain 1: S354C; heavy chain 2: Y349C).

In an initial experiment three different knob heavy chain to hole heavy chain ratios (K:H ratios) were transfected (SEQ ID NOs: 1, 2, 14): K:H=1:1, K:H=1.2:1 and K:H=1.5:1. In table 2 the IgG yields in the supernatants of the test expressions are shown.

TABLE 2
Fraction of full antibody, aggregates and incomplete antibody
in percent of the whole IgG yield of the VEGF-Her2-DAF test
expression (calculated via the percent area of each peak).

| | K:H = 1:1 | | K:H = 1.2:1 | | K:H = 1.5:1 | |
|---|---|---|---|---|---|---|
| | Repli- | Repli- | Repli- | Repli- | Repli- | Repli- |
| | cate 1 | cate 2 | cate 1 | cate 2 | cate 1 | cate 2 |
| antibody | 94.1 | 90.0 | 80.5 | 83.3 | 68.3 | 76.0 |
| aggregates | 5.9 | 10.0 | 9.1 | 4.3 | 5.9 | 4.1 |
| Incomplete | — | — | 10.3 | 12.4 | 25.8 | 19.9 |
| antibody | | | | | | |

For the VEGF-Her2-DAF parental the K:H=1.5:1 ratio showed the highest IgG concentration followed by the K:H=1:1 and K:H=1.2:1 (knob heavy chain to hole heavy chain) ratios. For the K:H=1.2:1 ratio the second replicate contained a very low IgG concentration. This was probably due to a lower viability of this batch of cells compared to the cells used in the first replicate of the expression. Analytical HPLC of the VEGF-Her2-DAF test expressions (Table 2, FIG. 6a) and the reducing and non-reducing SDS-PAGE (FIG. 6b) demonstrated the lowest side products and highest amount of the desired complete antibody for the K:H=1:1 ratio compared to the K:H ratios with increased knob heavy chain. The increase in knob chain correlated with an increase of incomplete antibodies. To accelerate the analysis of the test expressions all SDS-PAGEs were done with supernatants that had only been purified by sterile filtration and immunoprecipitation. Size-exclusion chromatography was omitted. The size of the complete antibody is 146 kDa, due to heavy chain glycosylation an apparent higher molecular weight is observed. In the analytical HPLC the main peak was eluted at about 8.8 min and corresponds to the expected size of the complete antibody (FIG. 6c). Thus, we could successfully produce VEGF-Her2-DAF with knobs-into-holes.

### Example 2

### Analysis of KiH VEGF-Her2 DAF-xAng2

After analysis of the VEGF-Her2-DAF had shown that the “knobs-into-holes” (KiH) concept did not interfere with the VEGF-Her2-DAF format we aimed to create a trispecific antibody by bringing together the DAF and the crossmab format in one antibody (FIG. 3a, b). The VEGF-Her2-DAF-xAng2 antibody was to this end expressed with three knob heavy chain to hole heavy chain ratios (K:H ratios) 1:1, 1.2:1 and 1.5:1 (SEQ ID NOs: 1, 5, 14, and 15). With increasing knob chain the IgG yield in the supernatant showed a decreasing trend (Table 3).

TABLE 3
Fraction of complete antibody, aggregates and incomplete antibody
in percent of the whole IgG yield of the VEGF-Her2-DAF-xAng2 test
expression (calculated via percent area of each peak).

| | K:H = 1:1 | | K:H = 1.2:1 | | K:H = 1.5:1 | |
|---|---|---|---|---|---|---|
| | Repli- | Repli- | Repli- | Repli- | Repli- | Repli- |
| | cate 1 | cate 2 | cate 1 | cate 2 | cate 1 | cate 2 |
| Antibody | 63.4 | 75.8 | 74.6 | 79.9 | 79.0 | 80.9 |
| Aggregates | 11.7 | 8.8 | 11.3 | 7.9 | 13.5 | 8.6 |
| Incomplete | 24.8 | 15.4 | 14.1 | 12.2 | 7.5 | 10.5 |
| antibody | | | | | | |

The analytical HPLC and SDS-PAGE analysis revealed that a K:H=1.5:1 gave the best product to side-product ratio. Increasing amounts of knob chain encoding plasmid led to less incomplete antibody as observed in the analytical HPLC (FIG. 7a and table 3). In the non-reducing SDS-PAGE of the K:H=1:1 and K:H=1.2:1 ratios (FIG. 7b) five bands indicate the complete antibody, antibody missing one light chain, two paired heavy chains, half an antibody and presumably two paired light chains (146 kDa, 122 kDa, 100 kDa, 73 kDa and 46 kDa, respectively, without glycosylation). The characterization is based on the theoretical molecular weight. The analytical HPLC confirms this finding with peaks corresponding to aggregates (6.6 min), complete antibody (8.8 min) and the presumed light chain dimer (10.5 min, and FIG. 7a). VEGF-Her2-DAF as well as VEGF-Her2-DAF-xAng2 expressed will in transient expressions and gave yields in the range of regular antibodies (Table 4).

TABLE 4
IgG concentration determined by Prot A
precipitation and HPLC quantitation.

| | | Concentration [μg/ml] | | |
|---|---|---|---|---|
| | Knob:Hole | Repli- | Repli- | Mean ± |
| Sample Name | Ratio | cate 1 | cate 2 | standard deviation |
| VEGF-Her2-DAF | 1:1 | 123.53 | 115.81 | 119.67 ± 3.86 |
| parental | 1.2:1 | 106.22 | (17.48) | 106.22 |
| | 1.5:1 | 140.49 | 140.06 | 140.23 ± 0.25 |
| VEGF-Her2-DAF- | 1:1 | 87.05 | 72.11 | 79.58 ± 7.47 |
| xAng2 | 1.2:1 | 78.93 | 66.47 | 72.70 ± 6.23 |
| | 1.5:1 | 63.78 | 63.04 | 63.41 ± 0.37 |

### Example 3

### Analysis of Kill Her2-VEGF DAF-xHer1-Her3 DAF

Furthermore, it is possible to combine two dual-affinity antibodies within one antibody format. With this approach it is essentially possible to generate tetraspecific antibodies with two Fab arms and a regular IgG backbone. The knobs-into-holes technology was used to differentiate the heavy chains and the Her1-Her3 dual affinity Fab arm was crossed by CH1-CL exchange between heavy and light chains (SEQ TD NOs: 1, 12, 14, 20). A fixed ratio of K:H of 1.2:1 was used for the heavy chains and a 1:1 ratio for the light chains. In the reducing gel the two different light chains can be differentiated (at approx. 25 kDa). The heavy chains fall together at about 50 kDa under reducing conditions. Under non-reducing conditions a slight smearing is observable for the full length antibody band (about 150 kDa) and a second prominent band is visible at about 110 kDa (FIG. 8a, b). As the analytical HPLC reveals a homogenous band this might be indicative of incomplete disulfide bridge formation. Additionally, it is of note that this was immunoprecipitation of supernatant without any prior size-exclusion purification. The mean raw expression values were for two independent biological expressions were 59.7 and 111.5 μg/ml.

### Example 4

### Analysis of KiH Her1-Her3 DAF-xHer2

In another example we generated a trispecific antibody which can bind to the ErbB family members HER1 (EGFR), HER2 (ErbB2) and HER3 (Her3). The knobs-into-holes technology was used to differentiate the heavy chains and the Her2 Fab arm was crossed by CH1-CL exchange between heavy and light chains (SEQ ID NOs: 4, 9, 13, 18). A fixed ratio of K:H of 1.2:1 was used for the heavy chains and a 1:1 ratio for the light chains. In the reducing gel the two different light chains can be differentiated (at approx. 25 kDa). Mean expression yield of this antibody in two independent expressions was 91.7 and 99.1 μg/mL.

### Example 5

### Proliferation Assay with KiH Her1-Her3 DAF-xHer2

The epidermoid cancer cell line A431 expresses high levels of EGFR, but also and HER2 and HER3 are expressed on A431 epidermoid cancer cells Inhibition of inter alia, EGFR is known to affect proliferation in this cell line. To evaluate efficacy of inter alia the EGFR part of the trispecific antibody KiH Her1-Her3 DAF-xHer2 (SEQ ID NOs: 4, 9, 13 and 18) a proliferation assay was performed with this cell line in the absence or presence of therapeutic antibody or a control IgG (JI, #015-000-003) antibody. 4000 cells were seeded per well of a 96-well cell culture plate in 100 μL growth medium supplemented with 1% fetal calf serum (FCS). The following day, 20 μL of serum reduced (1% FCS) medium was added containing therapeutic antibody to yield a final concentration of the antibody of 30 μg/mL. Cells were allowed to grow an additional five days upon which an ATP-release assay (Cell Titer Glow, Promega) was performed (FIG. 10a, b). Luminescence was recorded in a plate reader (TECAN). The trispecific antibody had a prominent anti-proliferative effect of 53.6+/−2.7% in this cell line (FIG. 10a).

### Example 6

### Proliferation Assay with KiH Her1-Her3 DAF-xHer2

The breast cancer cell line MDA-MB-175 VII expresses the ErbB family members Her2 and Her3 and harbors an autocrine heregulin loop. To evaluate efficacy of the Her2 and Her3 part of the trispecific antibody a proliferation assay was performed with this cell line. 20000 cells were seeded per well of a 96-well cell culture plate in 100 μL growth medium containing 10% FCS. The following day, 20 μL of full growth medium containing therapeutic antibody were added in a manner that the final antibody concentration equaled a dilution series (FIG. 11a). After additional five days of continued growth an ATP-release assay was performed (Cell Titer Glow, Promega). Luminesence was recorded in a plate reader (Tecan). The trispecific antibody inhibited growth in a dose-dependent manner and reached a maximal inhibition of 92.1+/−0.3% at 50 μg/mL (FIG. 11b).

### Example 7

### See Also (FIG. 12 a,b,c)

### Binding Kinetics of Kill Her1-Her3 DAF-xHer2

The binding kinetics of the trispecific antibody KiH Her1-Her3 DAF-xHer2 (SEQ ID NOs: 4, 9, 13 and 18) or of the respective parental antibodies was determined by surface plasmon resonance. To this end, in HEK-293F produced ErbB receptor ectodomains (ECD) were purified and used as analytes to determine affinities and simultaneous binding properties. The affinity data clearly showed comparable kinetic profiles for KiH Her1-Her3 DAF-xHer2 and the parental DAF and pertuzumab in their binding to Her1 ECD, Her2 ECD and Her3 ECD (Table 5).

TABLE 5
Binding kinetics measured by surface plasmon resonance at 37° C.

| | | ka | kd | t ½ | KD |
|---|---|---|---|---|---|
| ligand | analyte | [1/M*s] | [1/s] | [min] | [M] |
| Her1-Her3 DAF- | hu HER1 | 1.2E+06 | 2.0E−02 | 0.6 | 1.7E−08 |
| xHer2 | ECD | | | | |
| Her1-Her3 DAF- | hu HER2 | 1.9E+05 | 8.6E−04 | 13.5 | 4.4E−09 |
| xHer2 | ECD | | | | |
| Her1-Her3 DAF- | hu HER3 | 2.4E+06 | 4.4E−03 | 2.6 | 1.8E−09 |
| xHer2 | ECD | | | | |
| DAF | hu HER1 | 1.4E+06 | 2.1E−02 | 0.6 | 1.4E−08 |
| | ECD | | | | |
| Pertuzumab | hu HER2 | 2.0E+05 | 1.0E−03 | 11.2 | 5.0E−09 |
| | ECD | | | | |
| DAF | hu HER3 | 2.0E+06 | 3.7E−03 | 3.1 | 1.9E−09 |
| | ECD | | | | |

We next addressed the question whether the antigens could be bound simultaneously by consecutive injections of receptor ectodomains. In summary, we demonstrate that KiH Her1-Her3 DAF-xHer2 can simultaneously bind antigen combinations of Her1/Her2 or Her3/Her2. If injected in inversed order it was shown for the combination of Her2 and Her3 that KiH Her1-Her3 DAF-xHer2 can also bind simultaneously both antigens independent from the order of antigen injection. Pertuzumab binds, as expected, only Her2. The DAF antibody binds either Her1 or Her3, as expected (FIG. 12 a,b,c).

### Example 8

### See Also (FIG. 13)

### Tumor Cell Killing and ADCC Induction by Kill Her1-Her3 DAF-xHer2 Antibody in A431 Epidermoid Cancer Cells

For imaging the ADCC process and tumor cell killing of trispecific KiH Her1-Her3 DAF-xHer2 antibody (SEQ ID NOs: 4, 9, 13 and 18), A431 epidermoid carcinoma cells were grown on glass coverglasses and labelled with a green viability marker (CMFDA). Next, NK92natural killer cells that were stained with a red membrane stain (PKH26) were added on top of the tumor cells together with antibody KiH Her1-Her3 DAF-xHer2 directed against three Her members Her1, Her2 and Her3. Imaging was performed on a LEICA SP5× white light laser confocal microscope using a 63×/1.2NA water immersion lens on a heated stage supplying CO2 and humidity. Within minutes upon adding the antibody/NK cells, the killer cells start attacking the tumor. This is mediated by interacting via their FcγRIII (CD16) receptors with the tumor bound antibody. It can clearly be seen how cytolytic granules (releasing perforins and granzymes) are recruited towards the tumor cell surface which leads to a rapid lysis of the tumor cells as demonstrated by the loss of green fluorescence (=viability marker). Remarkable is the fierce and rapid attack that is mediated by the triple binding form of the antibody. Within 2.5h virtually the whole tumor mass has been eliminated. Results are shown in FIG. 13.

### Example 9

### Glycoengineered, Afucoyslated Trispecific Kill Her1-Her3 DAF-xHer2 Antibody (Amount of Fucose Between 5% and 65%) and In Vitro ADCC in KPL-4 or A431 Tumor Cells by 1 μg/ml specLysis %

The glycoengineered, afucosylated version of antibody KiH Her1-Her3 DAF-xHer2 (SEQ ID NOs: 4, 9, 13 and 18) is prepared by co-transfection with several plasmids, the ones for antibody expression, and one for a fusion GnTIII polypeptide expression (a GnT-III expression vector), and one for mannosidase II expression (a Golgi mannosidase II expression vector) at a ratio of 4 (antibody vectors):1 (GnT-III expression vector):1 (Golgi mannosidase II expression vector) in HEK293 or CHO cells.

The full antibody heavy and light chain DNA sequences were subcloned into mammalian expression vectors (one for the light chain and one for the heavy chain) under the control of the MPSV promoter and upstream of a synthetic polyA site, each vector carrying an EBV OriP sequence. Antibodies were produced by co-transfecting HEK293-EBNA cells or CHO cells with the antibody heavy and light chain expression vectors using a calcium phosphate-transfection approach. Exponentially growing HEK293-EBNA cells are transfected by the calcium phosphate method. For the production of the glycoengineered antibody, the cells are co-transfected with several plasmids, the ones for antibody expression, and one for a fusion GnTIII polypeptide expression (a GnT-III expression vector), and one for mannosidase II expression (a Golgi mannosidase II expression vector) at a ratio of 4 (antibody vectors):1 (GnT-III expression vector):1 (Golgi mannosidase II expression vector). Cells are grown as adherent monolayer cultures in T flasks using DMEM culture medium supplemented with 10% FCS, and are transfected when they are between 50 and 80% confluent. For the transfection of a T150 flask, 15 million cells are seeded 24 hours before transfection in 25 ml DMEM culture medium supplemented with FCS (at 10% V/V final), and cells are placed at 37° C. in an incubator with a 5% CO2 atmosphere overnight. For every antibody to be produced, a solution of DNA, CaCl2 and water is prepared by mixing 188 μg total plasmid vector DNA (several plasmids, the ones for antibody expression, and one for a fusion GnTIII polypeptide expression (a GnT-III expression vector), and one for mannosidase II expression (a Golgi mannosidase II expression vector) at a ratio of 4 (antibody vectors):1 (GnT-III expression vector):1 (Golgi mannosidase TI expression vector)), water to a final volume of 938 μl and 938 μl of a 1M CaCl2 solution. To this solution, 1876 μl of a 50 mM HEPES, 280 mM NaCl, 1.5 mM Na2HPO4 solution at pH 7.05 are added, mixed immediately for 10 sec and left to stand at room temperature for 20 sec. The suspension is diluted with 46 ml of DMEM supplemented with 2% FCS, and divided into two T150 flasks in place of the existing medium.

The cells are incubated at 37° C., 5% CO2 for about 17 to 20 hours, then medium is replaced with 25 ml DMEM, 10% FCS. The conditioned culture medium is harvested 7 days post-transfection by centrifugation for 15 min at 210×g, the solution is sterile filtered (0.22 μm filter) and sodium azide in a final concentration of 0.01% w/v is added, and kept at 4° C.

The secreted afucosylated antibodies are purified and the oligosaccharides attached to the Fc region of the antibodies were analysed e.g. by MALDI/TOF-MS (as described in e.g. WO 2008/077546). For this analysis oligosaccharides are enzymatically released from the antibodies by PNGaseF digestion, with the antibodies being either immobilized on a PVDF membrane or in solution. The resulting digest solution containing the released oligosaccharides is either prepared directly for MALDI/TOF-MS analysis or is further digested with EndoH glycosidase prior to sample preparation for MALDI/TOF-MS analysis. The analyzed amount of fucose within the sugar chain at Asn297 is between 65-5%.

The target cells (KPL4 breast carcinoma cells or A431 epidermoid cancer cells, cultivation in RPM11640+2 mM L-alanyl-L-Glutamine+10% FCS) are collected with trypsin/EDTA (Gibco #25300-054) in exponential growth phase. After a washing step and checking cell number and viability, the aliquot needed is labeled for 30 min at 37° C. in the cell incubator with calcein (Invitrogen #C3100MP; 1 vial is resuspended in 50 μl DMSO for 5 Mio cells in 5 ml medium). Afterwards, the cells are washed three times with AIM-V medium, the cell number and viability is checked and the cell number adjusted to 0.3 Mio/ml.

Meanwhile, PBMC (Peripheral Blood Mononuclear Cells) as effector cells are prepared by density gradient centrifugation (Histopaque-1077, Sigma # H8889) according to the manufacturer's protocol (washing steps 1× at 400 g and 2× at 350 g 10 min each). The cell number and viability is checked and the cell number adjusted to 15 Mio/ml.

100 μl calcein-stained target cells are plated in round-bottom 96-well plates, 50 μl diluted, afucosylated antibody (Mab205.10.1, Mab205.10.2, Mab205.10.3, preparation see below) which is added and 50 μl effector cells. In some experiments the target cells are mixed with Redimune NF Liquid (ZLB Behring) at a concentration of 10 mg/ml Redimune.

As controls serves the spontaneous lysis, determined by co-culturing target and effector cells without antibody and the maximal lysis, determined by 1% Triton X-100 lysis of target cells only. The plate is incubated for 4 hours at 37° C. in a humidified cell incubator.

The killing of target cells is assessed by measuring LDH (Lactate Dehydrogenase) release from damaged cells using the Cytotoxicity Detection kit (LDH Detection Kit, Roche #1 644 793) according to the manufacturer's instruction. Briefly, 100 μl supernatant from each well was mixed with 100 μl substrate from the kit in a transparent flat bottom 96 well plate. The Vmax values of the substrate's colour reaction is determined in an ELISA reader at 490 nm for at least 10 min. Percentage of specific antibody-mediated killing is calculated as follows: ((A−SR)/(MR−SR)×100, where A is the mean of Vmax at a specific antibody concentration, SR is the mean of Vmax of the spontaneous release and MR is the mean of Vmax of the maximal release.

As additional readout the calcein retention of intact target cells is assessed by lysing the remaining target cells in borate buffer (5 mM sodium borate+0.1% Triton) and measuring the calcein fluorescence in a fluorescence plate reader.

### Example 10

### In Vivo Antitumor Efficacy

The in vivo antitumor efficacy of antibody KiH Her1-Her3 DAF-xHer2 (SEQ ID NOs: 4, 9, 13 and 18) can be detected in cell and fragment based models of various tumor origin (e.g. lung cancer, SCCHN, breast- and pancreatic cancer) transplanted on SCID beige or nude mice. One example is the A431 epidermoid cancer cell xenograft model

A431 epidermoid cancer cells express HER1 and also HER2 and Her3 on the cell surface. A431 cells are maintained under standard cell culture conditions in the logarithmic growth phase. Ten million cells are engrafted to SCID beige mice. Treatment starts after tumors are established and have reached a size of 100-150 mm3. Mice are treated with e.g. a loading dose of 20 mg/kg of antibody/mouse and then once weekly with 10 mg/kg of antibody/mouse. Tumor volume is measured twice a week and animal weights are monitored in parallel.

1. A multispecific antibody, comprising:
A) a) the light chain and heavy chain of a full length antibody which specifically binds to a first antigen and second antigen; and
b) the modified light chain and modified heavy chain of a full length antibody which specifically binds to a third antigen, wherein the variable domains VL and VH are replaced by each other, and/or wherein the constant domains CL and CH1 are replaced by each other;
or
B) a) the light chain and heavy chain of a full length antibody which specifically binds to a first antigen; and
b) the modified light chain and modified heavy chain of a full length antibody which specifically binds to a second antigen and third antigen, wherein the variable domains VL and VH are replaced by each other, and/or wherein the constant domains CL and CH1 are replaced by each other;
or
C) a) the light chain and heavy chain of a full length antibody which specifically binds to a first antigen and a second antigen; and
b) the modified light chain and modified heavy chain of a full length antibody which specifically binds to a third antigen and fourth antigen, wherein the variable domains VL and VH are replaced by each other, and/or wherein the constant domains CL and CH1 are replaced by each other; and
wherein the antibody is a bivalent, tri- or tetraspecific antibody.

2. The multispecific antibody according to claim 1, wherein the antibody is trispecific and comprises
A) a) the light chain and heavy chain of a full length antibody which specifically binds to a first antigen and second antigen; and
b) the modified light chain and modified heavy chain of a full length antibody which specifically binds to a third antigen, wherein the variable domains VL and VH are replaced by each other, and/or wherein the constant domains CL and CH1 are replaced by each other;
or
B) a) the light chain and heavy chain of a full length antibody which specifically binds to a first antigen; and
b) the modified light chain and modified heavy chain of a full length antibody which specifically binds to a second antigen and third antigen, wherein the variable domains VL and VH are replaced by each other, and/or wherein the constant domains CL and CH1 are replaced by each other.

3. The multispecific antibody according to claim 2, wherein
under A) the first antigen is human HER1, the second antigen human HER3 and the third antigen is human HER2; or
under B) the first antigen is human HER2, the second antigen human HER1 and the third antigen is human HER3.

4. The multispecific antibody according to claim 1, wherein the antibody is a bivalent, trispecific antibody and comprises a
a) the light chain and heavy chain of a full length antibody which specifically binds to human HER1 and human HER3; and
b) the modified light chain and modified heavy chain of a full length antibody which specifically binds to human HER2, wherein the variable domains VL and VH are replaced by each other, and/or wherein the constant domains CL and CH1 are replaced by each other.

5. The multispecific antibody according to claim 4, wherein the antibody comprises the amino acid sequences of SEQ ID NOs: 4, 9, 13 and 18.

6. The multispecific antibody according to claim 3, wherein
under A) the first antigen is human HER1, the second antigen human HER3 and the third antigen is human cMET; or
under B) the first antigen is human cMET, the second antigen human HER1 and the third antigen is human HER3.

7. The multispecific antibody according to claim 1, wherein the antibody is tetraspecific and comprises
a) the light chain and heavy chain of a full length antibody which specifically binds to a first antigen and a second antigen; and
b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.

8. The multispecific antibody according to claim 1, wherein in the modified light chain and modified heavy chain under b) the variable domains VL and VH are replaced by each other; and wherein the constant domains CL and CH1 are replaced by each other.

9. The multispecific antibody according to claim 1, wherein in the modified light chain and modified heavy chain under b) (only) the variable domains VL and VH are replaced by each other.

10. The multispecific antibody according to claim 1, wherein in the modified light chain and modified heavy chain under b) (only) the constant domains CL and CH1 are replaced by each other.

11. The multispecific antibody according to claim 1, comprising
the CH3 domain of the heavy chain of the full length antibody of a) and
the CH3 domain of the modified heavy chain of the full length antibody of b) each meet at an interface which comprises an original interface between the antibody CH3 domains;
wherein said interface is altered to promote the formation of the trispecific or tetraspecific antibody, wherein the alteration is characterized in that:
i) the CH3 domain of one heavy chain is altered, so that within the original interface the CH3 domain of one heavy chain that meets the original interface of the CH3 domain of the other heavy chain within the tri- or tetraspecific antibody,
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and
ii) the CH3 domain of the other heavy chain is altered, so that within the original interface of the second CH3 domain that meets the original interface of the first CH3 domain within the tri- or tetraspecific antibody
an amino acid residue is replaced with an amino acid residue having a smaller side chain volume, thereby generating a cavity within the interface of the second CH3 domain within which a protuberance within the interface of the first CH3 domain is positionable.

12. The multispecific antibody according to claim 11, wherein the said amino acid residue having a larger side chain volume is selected from the group consisting of arginine (R), phenylalanine (F), tyrosine (Y), tryptophan (W) and said amino acid residue having a smaller side chain volume is selected from the group consisting of alanine (A), serine (S), threonine (T), valine (V).

13. The multispecific antibody according to claim 11, wherein both CH3 domains are further altered by the introduction of cysteine (C) as amino acid in the corresponding positions of each CH3 domain such that a disulfide bridge between both CH3 domains can be formed.

14. A method for the preparation of a multispecific antibody according to claim 1 comprising the steps of
a) transforming a host cell with vectors comprising nucleic acid molecules encoding
A) a) the light chain and heavy chain of a full length antibody which specifically binds to a first antigen and second antigen; and
b) the modified light chain and modified heavy chain of a full length antibody which specifically binds to a third antigen, wherein the variable domains VL and VH are replaced by each other, and/or wherein the constant domains CL and CH1 are replaced by each other;
or
B) a) the light chain and heavy chain of a full length antibody which specifically binds to a first antigen; and
b) the modified light chain and modified heavy chain of a full length antibody which specifically binds to a second antigen and third antigen, wherein the variable domains VL and VH are replaced by each other, and/or wherein the constant domains CL and CH1 are replaced by each other;
or
C) a) the light chain and heavy chain of a full length antibody which specifically binds to a first antigen and a second antigen; and
b) the modified light chain and modified heavy chain of a full length antibody which specifically binds to a third antigen and fourth antigen, wherein the variable domains VL and VH are replaced by each other, and/or wherein the constant domains CL and CH1 are replaced by each other.
b) culturing the host cell under conditions that allow synthesis of said antibody molecule; and
c) recovering said antibody molecule from said culture.

15. Nucleic acid encoding the multispecific antibody according to claim 1.

16. Vectors comprising nucleic acid encoding the multispecific antibody according to claim 1.

17. A host cell comprising the vectors according to claim 16.

18. (canceled)

19. A pharmaceutical composition comprising an antibody according to claim 1 and at least one pharmaceutically acceptable excipient.

20. An antibody according to claim 1 for use in the treatment of cancer.

21. (canceled)

22. A method for the treatment of a patient in need of therapy, comprising administering to the patient a therapeutically effective amount of an antibody according to claim 1.

23. A method for the treatment of a patient suffering from cancer, comprising administering to the patient a therapeutically effective amount of an antibody according to claim 1.


SEQUENCE LISTING (20 sequences)

SEQ ID NO: 1 - protein, 469 aa, Artificial
  HC/knob/Her2/VEGF
MGWSCIILFLVATATGVHSEVQLVESGGGLVQPGGSLRLSCAASGFNISGTYIHWVRQAPGKGLEWVARIYPSEGYTRYADSVKGRFTISADTSKNTAYLQMNSLRAEDTAVYYCSRWVGVGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 469 aa, Artificial
  HC/hole/Her2/VEGF
MGWSCIILFLVATATGVHSEVQLVESGGGLVQPGGSLRLSCAASGFNISGTYIHWVRQAPGKGLEWVARIYPSEGYTRYADSVKGRFTISADTSKNTAYLQMNSLRAEDTAVYYCSRWVGVGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 470 aa, Artificial
  HC/knob/Her1/Her3
MGWSCIILFLVATATGVHSEVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 470 aa, Artificial
  HC/hole/Her1/Her3
MGWSCIILFLVATATGVHSEVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 482 aa, Artificial
  HC/hole/xAng2
MGWSCIILFLVATATGVHSQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 482 aa, Artificial
  HC/knob/xAng2
MGWSCIILFLVATATGVHSQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 471 aa, Artificial
  HC/hole/xIGF1R
MEFGLSWVFLVALLRGVQCQVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 473 aa, Artificial
  HC/hole/xHer3
MGWSCIILFLVATATGVHSQVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 472 aa, Artificial
  HC/hole/xHer2
MGWSCIILFLVATATGVHSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 472 aa, Artificial
  HC/hole/xcMet
MEFGLSWVFLVALLRGVQCEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 714 aa, Artificial
  HC/hole/scFabIGF1R
MGWSCIILFLVATATGVHSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 474 aa, Artificial
  HC/hole/xHer1/Her3
MGWSCIILFLVATATGVHSEVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 233 aa, Artificial
  LC/Her1/Her3
MGWSCIILFLVATATGVHSDIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 237 aa, Artificial
  LC/Her2/VEGF
MGWSCIILFLVATATGVHSDIQMTQSPSSLSASVGDRVTITCRASQNIAKTISGYVAWYQQKPGKAPKLLIYWGSFLYSGVPSRFSGSRSGTDFTLTISSLQPEDFATYYCQQHYSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 232 aa, Artificial
  LC/xAng2
MGWSCIILFLVATATGVHSQPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 16 - protein, 233 aa, Artificial
  LC/xIGF1R
MEAPAQLLFLLLLWLPDTTGEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 17 - protein, 237 aa, Artificial
  LC/xHer3
MGWSCIILFLVATATGVHSDIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 231 aa, Artificial
  LC/xHer2
MGWSCIILFLVATATGVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 237 aa, Artificial
  LC/xcMet
MGWSCIILFLVATATGVHSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 231 aa, Artificial
  LC/xHer1/Her3
MGWSCIILFLVATATGVHSDIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC